(19) United States
(12) Patent Application Publication
(10) Pub. No.: US 2023/0329970 A1
(43) Pub. Date: Oct. 19, 2023

(54) SYRINGE SYSTEM

(71) Applicant: KYOCERA Corporation, Kyoto-shi, Kyoto (JP)

(72) Inventors: Hisashi SAKAI, Kyoto-shi, Kyoto (JP); Yutaka MORI, Kyoto-shi, Kyoto (JP)

(21) Appl. No.: 18/033,255

(22) PCT Filed: Oct. 8, 2021
(86) PCT No.: PCT/JP2021/037347

§ 371 Date: Apr. 21, 2023

(30) Foreign Application Priority Data
Oct. 23, 2020 (JP) 2020-178279

(51) Int. Cl. A61J 1/05; A61B 5/153; A61B 5/15

(52) U.S. Cl. CPC A61J 1/05; A61B 5/153; A61B 5/150389; A61M 5/3129

(57) ABSTRACT

A syringe system includes a first syringe, a second syringe, and at least any one of a first buoy located inside a first syringe barrel or a second buoy located inside a second syringe barrel. The first syringe includes the first syringe barrel. The second syringe includes the second syringe barrel coupled to the first syringe in such a manner that at least part of the second syringe barrel is movable from inside the first syringe barrel.

### TECHNICAL FIELD

The present disclosure relates to a syringe system for preparing a liquid containing a desired component by fractionating blood or the like.

### BACKGROUND OF INVENTION

A buoy type suspension fractionation system for fractionating blood or the like using a specific gravity difference is known.

### SUMMARY

A syringe system according to an aspect of the present disclosure includes a first syringe having a first syringe barrel; a second syringe having a second syringe barrel coupled to the first syringe in a movable manner from inside the first syringe barrel; and at least one of a first buoy located inside the first syringe barrel or a second buoy located inside the second syringe barrel.

### BRIEF DESCRIPTION OF THE DRAWINGS

FIG. 1 is a diagram illustrating a configuration example of a preparation kit including a syringe system according to a first embodiment of the present disclosure.

FIG. 2 is a cross-sectional view illustrating an example of a first buoy and a second buoy including a plurality of buoy members.

FIG. 3 is a diagram illustrating an example of a shape of a buoy.

FIG. 4 is a diagram illustrating an example of a shape of a buoy.

FIG. 5 is a diagram illustrating an example of a shape of a buoy.

FIG. 6 is a diagram illustrating an example of a shape of the first buoy capable of functioning as a cover configured to block movement of a liquid.

FIG. 7 is a diagram illustrating another example of the shape of the first buoy capable of functioning as a cover configured to block movement of a liquid.

FIG. 8 is a diagram illustrating a configuration example of a preparation kit including a syringe system with the second buoy having two buoys.

FIG. 9 is a diagram illustrating a configuration example of a preparation kit including a syringe system with the first buoy having two buoys.

FIG. 10 is a diagram illustrating a configuration example of a preparation kit including a syringe system in which a second syringe can be coupled to a first gasket.

FIG. 11 is a diagram illustrating an example of a structure adopted for coupling a first attachment hole and a second port.

FIG. 12 is a diagram illustrating a configuration example of a preparation kit including a syringe system according to a second embodiment of the present disclosure.

FIG. 13 is a diagram illustrating a configuration example of a preparation kit including a syringe system in which a third syringe can be coupled to a second gasket.

FIG. 14 is a diagram illustrating a configuration example of a preparation kit including a syringe system having a third buoy.

FIG. 15 is a diagram illustrating an example of a process flow for preparing PRP from blood by using the preparation kit illustrated in FIG. 9.

FIG. 16 is a diagram illustrating an example of a process flow for preparing PRP from blood by using the preparation kit illustrated in FIG. 10.

FIG. 17 is a diagram illustrating an example of the first half of a process flow for preparing PRP from blood by using the preparation kit illustrated in FIG. 12.

FIG. 18 is a diagram illustrating an example of the second half of the process flow for preparing PRP from blood by using the preparation kit illustrated in FIG. 12.

FIG. 19 is a diagram illustrating an example of the first half of a process flow for preparing PRP from blood by using the preparation kit illustrated in FIG. 12.

FIG. 20 is a diagram illustrating the second half of the process flow for preparing PRP from blood by using the preparation kit illustrated in FIG. 12.

FIG. 21 is a diagram illustrating an example of a process flow for preparing PRP from blood by using a preparation kit including one first buoy and one second buoy.

FIG. 22 is a diagram illustrating another example of a process flow for preparing PRP from blood by using the preparation kit illustrated in FIG. 13.

FIG. 23 is a diagram illustrating another example of a process flow for preparing PRP from blood by using the preparation kit illustrated in FIG. 13.

FIG. 24 is a diagram illustrating an example of a process flow for preparing PRP from blood by using the preparation kit illustrated in FIG. 14.

### DESCRIPTION OF EMBODIMENTS

### First Embodiment

There is a growing interest in techniques and methods for fractionating blood or the like of a patient to prepare a liquid containing desired components. For example, in regenerative medicine, the usage of platelet rich plasma (hereinafter referred to as PRP) prepared from blood has attracted attention. In the present specification, “fractionation” refers to a process of separating a liquid into two or more layers. “Fractionation” means, for example, a process of separating a liquid into two or more layers in which the mass per unit volumes of the main components contained in the respective layers are different from each other, or a process of separating a liquid into two or more layers of liquids different from each other.

An embodiment of the present disclosure will be described in detail below. Hereinafter, a syringe system 100 configured to prepare platelet rich plasma (hereinafter, referred to as PRP) from blood will be described as an example. However, the present disclosure is not limited thereto, and the syringe system 100 may be used in a process of separating components based on the specific gravity of the components contained in various liquids. The syringe system 100 may be used for, for example, a process of separating a solution containing stem cells from bone marrow tissue or adipose tissue, in addition to the adjustment of PRP.

Configuration of Syringe System 100

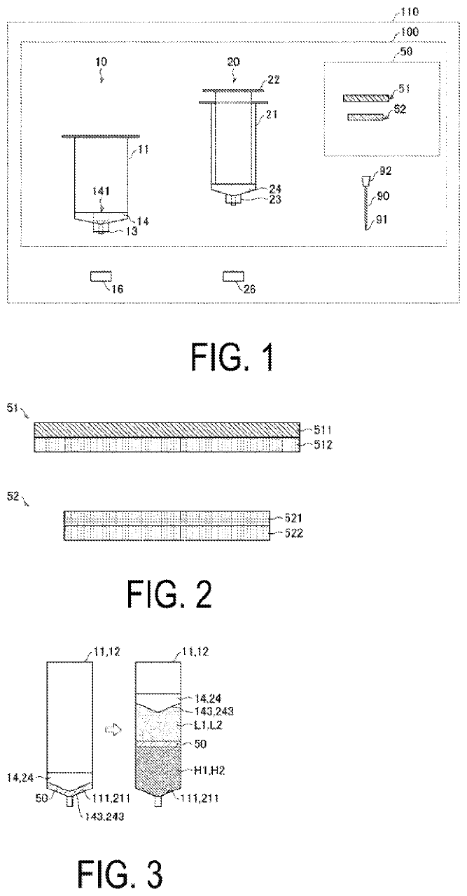

First, a configuration of the syringe system 100 according to a first embodiment of the present disclosure will be described using FIG. 1. FIG. 1 is a diagram illustrating a configuration example of a preparation kit 110 including the syringe system 100.

As illustrated in FIG. 1, the syringe system 100 includes a first syringe 10 having a first syringe barrel 11 capable of holding a liquid, a second syringe 20 having a second syringe barrel 21 capable of holding a liquid, and a buoy 50. The buoy 50 includes at least one of a first buoy 51 located inside the first syringe barrel 11 or a second buoy 52 located inside the second syringe barrel 21. The first buoy 51 is a buoy with respect to a liquid to be fractionated that is held in the first syringe barrel 11, and the second buoy 52 is a buoy with respect to a liquid to be fractionated that is held in the second syringe barrel 21. In FIG. 1, the syringe system 100 further including a first needle 90 is illustrated, but the first needle 90 is not an essential constituent element in the syringe system 100.

First Syringe 10

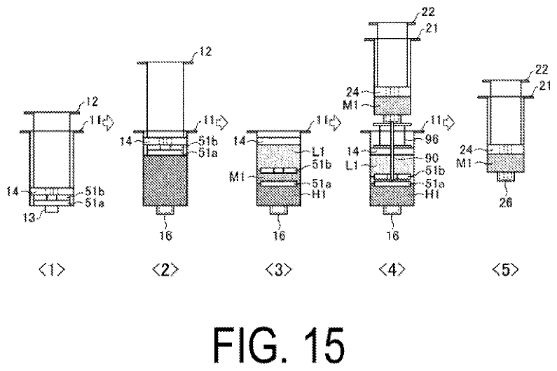

The first syringe 10 will be described first. The first syringe 10 may include a first gasket 14 positionable in the first syringe barrel 11. The first syringe 10 may include a first plunger 12 (see FIG. 15 and the like) detachably provided to the first gasket 14.

The first syringe barrel 11 can hold the stored liquid. The first syringe barrel 11 constitutes at least part of the first syringe 10 along with the first gasket 14. A first port 13 (first leading end portion) is disposed at one end portion of the first syringe barrel 11. A portion of the first syringe barrel 11 for holding the liquid may have a substantially cylindrical shape.

The liquid held in the first syringe barrel 11 may be any liquid containing platelets. As an example, the liquid held in the first syringe barrel 11 may be (1) blood, (2) bone marrow liquid, (3) liquid containing platelets collected from blood/bone marrow liquid/spleen or the like, (4) liquid containing platelets prepared in vitro, or the like.

The first syringe barrel 11 may be used as a centrifugation container. In the centrifugation processing, a heavy layer containing a component whose mass per unit volume is heavy (in other words, the density is high) and a light layer containing a component whose mass per unit volume is light (in other words, the density is low) are fractionated using centrifugal force, whereby the heavy layer and the light layer are separated from each other. In the present specification, high density may be expressed as large specific gravity, and low density may be expressed as small specific gravity.

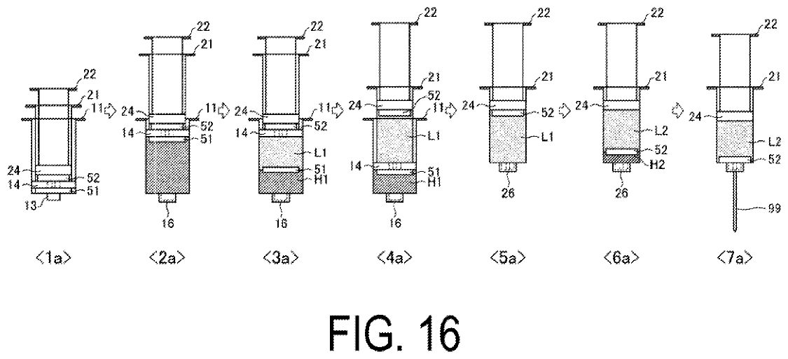

As an example, blood stored in the first syringe barrel 11 (that is, whole blood) is fractionated into a heavy layer H1 mainly containing red blood cells and a light layer L1 containing white blood cells and platelets by the centrifugation processing (see FIG. 16 and the like). This is because the density of red blood cells (about 1.102 g/ml) is higher than the density of white blood cells (1.064 to 1.097 g/ml), the density of platelets (1.03 to 1.04 g/ml), and the density of plasma (1.025 to 1.029 g/ml).

Alternatively, the blood stored in the first syringe barrel 11 may be fractionated into the heavy layer H1 mainly containing red blood cells, an intermediate layer M1 containing PRP, and the light layer L1 (PPP layer) by the centrifugation processing (see FIG. 15 and the like).

Herein, specific gravity means a ratio of the density of a subject (for example, red blood cells, white blood cells, platelets, or the buoy 50) to the density of a standard subject (for example, distilled water at four degrees Celsius). In the present specification, density means mass per unit volume. The density may be determined, for example, by the immersion method using a pycnometer (ISO 1183-1: 2019). When the mass per unit volume of a certain subject X is heavier than the mass per unit volume of a certain subject Y, this is expressed as the density of the subject X being higher than the density of the subject Y or the specific gravity of the subject X being larger than the specific gravity of the subject Y in the present specification.

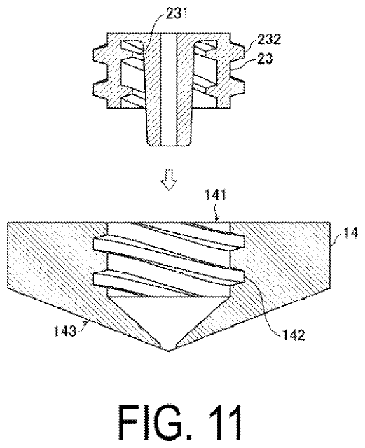

The first port 13 has a cylindrical shape. The internal space of the first port 13 communicates with the internal space of the first syringe barrel 11. The first port 13 may also be able to function as an attachment portion to which a blood-collecting tube for storing a liquid in the first syringe barrel 11, an injection needle (for example, a blood-collecting needle or a bone marrow needle), and the like are attached. For example, on an inner side surface of the first port 13, there may be formed a female screw to be engaged with a male screw formed on a blood-collecting tube (not illustrated) for collecting blood or bone marrow liquid. The female screw formed on the inner side surface of the first port 13 may be the same as a female screw 231 formed on an inner side surface of a second port 23 to be described later (see FIG. 11).

The first gasket 14 is located inside the first syringe barrel 11 and is moved in a reciprocating manner in the first syringe barrel 11. A first attachment hole 141, in which the first plunger 12 can be attached, is formed in the first gasket 14. The reciprocating movement of the first gasket 14 in the first syringe barrel 11 may be performed via the first plunger 12, for example. The reciprocating movement of the first gasket 14 makes it possible to introduce a liquid into or discharge the liquid from the first syringe barrel 11.

As illustrated in FIG. 1, the preparation kit 110 may include a first cap 16 capable of being attached in a liquid-tight manner to the first port 13. The first cap 16 is attached to the first port 13, for example, when the first syringe barrel 11 is used as a centrifugation container.

When the first cap 16 is attached to the first port 13, the first syringe barrel 11 may also function as a centrifugation container. The use of the first cap 16 makes it possible to perform centrifugation processing on a liquid while the liquid is being stored in the first syringe barrel 11, or to perform centrifugation processing while the first plunger 12 or the like is being attached in the first attachment hole 141.

Second Syringe 20

The second syringe 20 will be described below. The second syringe 20 may include a second gasket 24 positionable in the second syringe barrel 21. The second syringe 20 may include a second plunger 22 detachably provided to the second gasket 24.

The second syringe barrel 21 can hold the stored liquid. The second syringe barrel 21 constitutes at least part of the second syringe 20 along with the second gasket 24. A second port 23 (second leading end portion) is disposed at one end portion of the second syringe barrel 21. A portion of the second syringe barrel 21 for holding the liquid may have a substantially cylindrical shape.

In an example, the liquid stored and held in the second syringe barrel 21 may be part of the liquid held in the first syringe barrel 11.

The second syringe barrel 21 may be used as a centrifugation container. For example, when a liquid containing white blood cells and platelets is stored in the second syringe barrel 21, the liquid may be fractionated by centrifugation processing into a heavy layer H2 (that is, a layer containing PRP) having a high concentration of platelets, and a light layer L2 (that is, platelet poor plasma, and hereinafter referred to as PPP) having a low concentration of platelets (see FIG. 18 and the like).

In the present disclosure, when it is described that the concentration of each blood component is high or low without specifying a subject to be compared with, it means that the concentration of each blood component is high or low in comparison with whole blood. For example, the description “the concentration of platelets is high” means that the concentration of platelets is high in comparison with whole blood, and the description “the concentration of platelets is low” means that the concentration of platelets is low in comparison with whole blood. In the present disclosure, PRP refers to a liquid having a high concentration of platelets in comparison with whole blood. In the present disclosure, PRP may refer to a liquid having a high concentration of growth factors released from platelets in comparison with whole blood. Herein, as an index of the concentration of each blood component in the liquid, for example, the number of pieces of predetermined components per unit volume (pieces/μL) may be used. Each blood component in the liquid may be measured by, for example, an electrical resistance method or a flow cytometry method.

The second port 23 has a cylindrical shape. The internal space of the second port 23 communicates with the internal space of the second syringe barrel 21. The second port 23 may function as an attachment portion to which members (for example, a tube and an injection needle) for storing a liquid in the second syringe barrel 21 or discharging the liquid from the second syringe barrel 21 are attached. For example, the second port 23 may have a structure the same as or similar to that of the first port 13. That is, on an inner side surface of the second port, the female screw 231 to be engaged with a male screw of the injection needle may be formed (see FIG. 11). The male screw of the injection needle and the female screw 231 may be engaged with each other by a Luer lock scheme (ISO80369-7: 2016).

The second gasket 24 is located inside the second syringe barrel 21 and is moved in a reciprocating manner in the second syringe barrel 21. A second attachment hole (not illustrated) in which the second plunger 22 can be attached may be formed in the second gasket 24. The reciprocating movement of the second gasket 24 in the second syringe barrel 21 may be performed via the second plunger 22, for example. The reciprocating movement of the second gasket 24 makes it possible to introduce a liquid into or discharge a liquid from the second syringe barrel 21.

As illustrated in FIG. 1, the preparation kit 110 may include a second cap 26 capable of being attached in a liquid-tight manner to the second port 23. The second cap 26 is attached to the second port 23, for example, when the second syringe barrel 21 is used as a centrifugation container. The use of the second cap 26 makes it possible to perform centrifugation processing on a liquid in a state in which the liquid is stored in the second syringe barrel 21.

First Needle 90

The first needle 90 is a tubular needle having a first end 91 insertable into the first gasket 14 positionable in the first syringe barrel 11 and a second end 92 located on the opposite side to the first end 91. The first needle 90 may be coupled to the second port 23 of the second syringe barrel 21. When the second end 92 of the first needle 90 is coupled to the second port 23 and the first end 91 of the first needle 90 penetrates the first gasket 14 located inside the first syringe barrel 11, the second syringe 20 may be coupled to the first gasket 14 via the first needle 90. The first needle 90 may be used when part of the liquid (for example, the light layer L1) in the first syringe barrel 11 is moved into the second syringe barrel 21.

The preparation kit 110 may further include a first needle guide 96 used when the first end 91 of the first needle 90 penetrates the first gasket 14 (see FIG. 15). The first needle guide 96 includes a first guide hole serving as a through hole through which the first end 91 of the first needle 90 can move toward the first gasket 14. The first end 91 of the first needle 90 moves through the first guide hole and reaches the first gasket 14. The first end 91 having passed through the first guide hole penetrates the first gasket 14 and reaches the internal space in the first syringe barrel 11 (see FIG. 15 and the like).

Buoy 50

The buoy 50 will be described. The buoy 50 is required to include at least one of the first buoy 51 located inside the first syringe barrel 11 or the second buoy 52 located inside the second syringe barrel 21. The first buoy 51 is a buoy for the liquid in the first syringe barrel 11. That is, the first buoy 51 is a buoy located at a boundary portion between two or more layers when the liquid in the first syringe barrel 11 is fractionated into two or more layers. Likewise, the second buoy 52 is a buoy for the liquid in the second syringe barrel 21. That is, the second buoy 52 is a buoy located at a boundary portion between two or more layers when the liquid in the second syringe barrel 21 is fractionated into two or more layers. In an example, the first buoy 51 may be located at a boundary portion between the heavy layer H1 and the light layer L1 when the liquid in the first syringe barrel 11 is fractionated. In other words, the first buoy 51 may have a specific gravity smaller than the specific gravity of the heavy layer H1 when the liquid in the first syringe barrel 11 is fractionated and larger than the specific gravity of the light layer L1 when the liquid in the first syringe barrel 11 is fractionated. The second buoy 52 may be located at a boundary portion between the heavy layer H2 and the light layer L2 when the liquid in the second syringe barrel 21 is fractionated. In other words, the second buoy 52 may have a specific gravity smaller than the specific gravity of the heavy layer H2 when the liquid in the second syringe barrel 21 is fractionated and larger than the specific gravity of the light layer L2 when the liquid in the second syringe barrel 21 is fractionated.

For example, in a state in which the liquid and the first buoy 51 are stored in the first syringe barrel 11, when the liquid is fractionated into the heavy layer H1 having a large specific gravity and the light layer L1 having a small specific gravity, the first buoy 51 may be located at the boundary portion between the heavy layer H1 and the light layer L1.

For example, in a state in which the liquid and the second buoy 52 are stored in the second syringe barrel 21, when the liquid is fractionated into the heavy layer having a large specific gravity and the light layer having a small specific gravity, the second buoy 52 may be located at the boundary portion between the heavy layer H2 and the light layer L2.

FIG. 1 illustrates the syringe system 100 including the buoy 50 having the first buoy 51 and the second buoy 52, but the syringe system 100 is not limited to this configuration. That is, the buoy 50 of the syringe system 100 may include only the second buoy 52 or may include only the first buoy 51.

In the present specification, when the individual buoys such as the first buoy 51 and the second buoy 52 are not distinguished, these buoys may intentionally be referred to as the buoy 50. In the present specification, the buoy 50 means, in an example, the first buoy 51 and/or the second buoy 52.

The buoy 50 has a predetermined specific gravity. The buoy 50 may include two or more buoy members having different specific gravities. The buoy 50 may include, but is not limited to, at least any of buoy members made of polystyrene, polypropylene, polyethylene, silicone, acrylic resin, acrylonitrile-butadiene-styrene resin (ABS), acrylonitrile-styrene resin (AS), and combinations thereof.

FIG. 2 is a cross-sectional view illustrating an example of the first buoy 51 and the second buoy 52 including a plurality of buoy members. As illustrated in FIG. 2, the first buoy 51 may be formed by layering a first buoy member 511 and a second buoy member 512 having different specific gravity in a layered form. The first buoy member 511 and the second buoy member 512 may be layered in a layered form by being bonded to each other. In an example, the first buoy member 511 may be made of silicone and the second buoy member 512 may be made of polystyrene.

On the other hand, the second buoy 52 may be formed by bonding and layering a third buoy member 521 and a fourth buoy member 522 having different specific gravities in a layered form. In an example, the third buoy member 521 may be made of ABS and the second buoy member 512 may be made of silicone.

FIG. 2 illustrates an example of the first buoy 51 and the second buoy 52 each including two buoy members, but the present disclosure is not limited thereto. For example, the first buoy 51 and the second buoy 52 may each include a single buoy member or include three or more buoy members.

The first buoy 51 and the second buoy 52 may be made to have a predetermined specific gravity. As an example, the first buoy 51 and the second buoy 52 may include a plurality of layers of buoy members as illustrated in FIG. 2. Note that the first buoy 51 and the second buoy 52 illustrated in FIG. 2 are merely examples, and the first buoy 51 and the second buoy 52 each having a desired specific gravity may be realized by optionally combining one or more buoy members having different specific gravities.

In order to prepare PRP from whole blood, for example, the following two centrifugation processing operations may be performed.

First centrifugation processing: by using the first syringe barrel 11 as a centrifugation container, whole blood stored in the first syringe barrel 11 is fractionated into the heavy layer H1 and the light layer L1 by centrifugal force, and the light layer L1 is separated by being moved into the second syringe barrel 21 or the like (see FIG. 16 and the like).
Second centrifugation processing: by using the second syringe barrel 21 as a centrifugation container, the light layer L1 having been separated by the first centrifugation processing and stored in the second syringe barrel 21 is fractionated into the heavy layer H2 (the layer containing PRP) and the light layer L2 (PPP layer) by centrifugal force, and the heavy layer H2, for example, is separated by being taken out from the second syringe barrel 21 (see FIG. 16 and the like).

The first buoy 51 may have specific gravity larger than the specific gravity of whole blood of a human (1.052 to 1.060 for men and 1.049 to 1.056 for women). For example, it may have specific gravity larger than 1.049 g/ml, or may have specific gravity larger than 1. 052 g/ml. For example, the first buoy 51 may have a density between the density of red blood cells (about 1.102 g/ml) and the density of white blood cells (1.064 to 1.097 g/ml). For example, the density of the first buoy 51 may be 1.07 to 1.10 g/ml, or may be 1.09 g/ml. The first buoy 51 having such a density is located at the boundary portion between the heavy layer H1 and the light layer L1 in the first syringe barrel 11 after the first centrifugation processing. This makes it possible to selectively store the liquid of the light layer L1 into the second syringe barrel 21 while reducing contamination by red blood cells.

On the other hand, the mass per unit volume of the second buoy 52 may be equal to or less than the mass per unit volume of the first buoy 51. The second buoy 52 may have a density between the density of platelets (1.03 to 1.04 g/ml) and the density of plasma (1.025 to 1.029 g/ml). For example, the density of the second buoy 52 may be 1.026 to 1.05 g/ml, or may be 1.03 g/ml. The second buoy 52 having such a density is located at the boundary portion between the heavy layer H2 and the light layer L2 in the second syringe barrel 21 when the fractionation is carried out in the second centrifugation processing. This makes it possible to selectively collect the heavy layer H2 containing PRP while reducing the mixing of plasma. For example, the second buoy 52 may have a specific gravity between the density of white blood cells (1.064 to 1.097 g/ml) and the density of platelets (1.03 to 1.04 g/ml). For example, the density of the second buoy 52 may be 1.04 to 1.08 g/ml, or may be 1.05 g/ml. The second buoy 52 having such density is located at the boundary portion between the heavy layer H2 and the light layer L2 in the second syringe barrel 21 after the second centrifugation processing. This makes it possible to selectively collect the light layer L2 containing PRP while reducing contamination by white blood cells.

The buoy 50 may have any of a through hole, a net-like portion, and a nonwoven fabric-like portion in at least one location. For example, a through hole, a net-like portion, and a nonwoven fabric-like portion may be provided in a center portion of the buoy 50. Alternatively, the entire buoy 50 may be a net-like portion or a nonwoven fabric-like portion. Herein, the term “nonwoven fabric” refers to a fabric produced by mechanically, chemically, and thermally treating fiber sheets (webs) and bonding them with an adhesive, a fusing force of the fiber itself, or the like. The nonwoven fabric-like portion may be a site where the structure of the nonwoven fabric is achieved using the above-mentioned buoy members and the like. This configuration may cause the buoy 50 not to obstruct the movement of each component of the liquid in the first syringe barrel 11 in the first centrifugation processing and the movement of each component of the liquid in the second syringe barrel 21 in the second centrifugation processing.

The shape of the buoy 50 will be described next by using FIGS. 3 to 5. Each of FIGS. 3 to 5 is a diagram illustrating an example of the shape of the buoy 50. Note that FIGS. 3 to 5 are diagrams for illustrating the shape of the buoy 50, and the sizes of the illustrated members are not accurate. In FIGS. 3 to 5, in a case in which the illustrated buoy 50 is the first buoy 51, the first syringe barrel 11 and the first gasket 14 are illustrated. On the other hand, in a case in which the illustrated buoy 50 is the second buoy 52, the second syringe barrel 21 and the second gasket 24 are illustrated.

In FIGS. 3 to 5, on the left side, the first syringe barrel 11 (or the second syringe barrel 21) storing no liquid therein and the first buoy 51 (or the second buoy 52) are illustrated. On the other hand, on the right side, the first buoy 51 (or the second buoy 52) is illustrated in a state where the liquid in the first syringe barrel 11 (or the second syringe barrel 21) has been fractionated in the centrifugation processing.

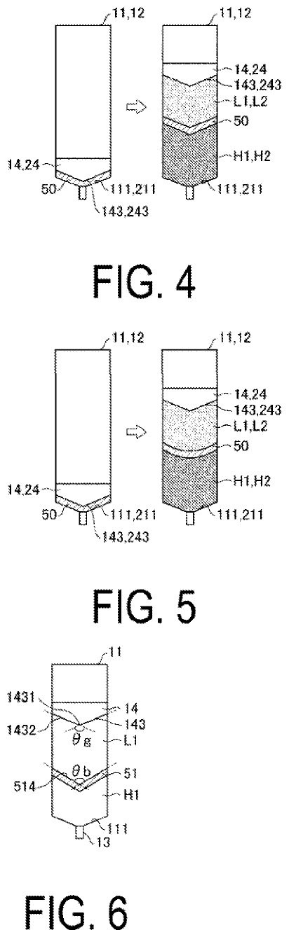

At least any one of the first buoy 51 or the second buoy 52 may have a substantially circular flat plate shape, a disk shape having a conical surface in which the center portion protrudes relative to the peripheral edge portion, or a disk shape having a curved surface in which the center portion protrudes relative to the peripheral edge portion. FIG. 3 illustrates an example of the buoy 50 having a substantially circular flat plate shape. FIG. 4 illustrates an example of the buoy 50 having a disk shape with a conical surface in which the center portion protrudes relative to the peripheral edge portion. FIG. 5 illustrates an example of the buoy 50 having a disk shape with a curved surface in which the center portion protrudes relative to the peripheral edge portion.

The diameter of the first buoy 51 is desirably smaller than the inner diameter of the first syringe barrel 11, and the diameter of the second buoy 52 may be smaller than the inner diameter of the second syringe barrel 21. As a result, the first buoy 51 and the second buoy 52 are likely to move smoothly in the first syringe barrel 11 and the second syringe barrel 21, respectively. However, the present disclosure is not limited thereto as long as the first buoy 51 and the second buoy 52 can move smoothly in the first syringe barrel 11 and the second syringe barrel 21, respectively.

The buoy 50 has a first point P1 at any position on the lower surface and a second point P2 at any position on the upper surface. For example, the length of at least one of the straight lines connecting the first point P1 and the second point P2 of the first buoy 51 may be larger than the inner diameter of the first syringe barrel 11. This makes it possible to suppress a changeover of the upper and lower surfaces of the first buoy 51 due to the rotation of the first buoy 51 in the first syringe barrel 11. In addition, for example, the length of at least one of the straight lines connecting the first point P1 and the second point P2 of the second buoy 52 may be larger than the inner diameter of the second syringe barrel 21. This makes it possible to suppress a changeover of the upper and lower surfaces of the second buoy 52 due to the rotation of the second buoy 52 in the second syringe barrel 21. Herein, the lower surface of the first buoy 51 is a surface facing a bottom surface 111 of the first syringe barrel 11, and the upper surface of the first buoy 51 is a surface on the opposite side to the lower surface of the first buoy 51. The bottom surface 111 of the first syringe barrel 11 is a surface of the deepest portion in the first syringe barrel 11, and is also a surface in which a communication hole between the internal space of the first syringe barrel 11 and the internal space of the first port 13 is provided in an example. On the other hand, the upper surface of the second buoy 52 is a surface on the opposite side to the lower surface of the second buoy 52. A bottom surface 211 of the second syringe barrel 21 is a surface of the deepest portion in the second syringe barrel 21, and is also a surface in which a communication hole between the internal space of the second syringe barrel 21 and the internal space of the second port 23 is provided in an example.

The buoy 50 may have flexibility. For example, the first buoy 51 is sandwiched between a lower surface 143 of the first gasket 14 and the bottom surface 111 in the first syringe barrel 11 in a state in which no liquid is stored in the first syringe barrel 11 and the first gasket 14 is pushed to the deepest portion in the first syringe barrel 11. In this case, as illustrated in FIGS. 3 to 5, the first buoy 51 may be deformed in accordance with the shape of a gap between the lower surface 143 of the first gasket 14 and the bottom surface 111 in the first syringe barrel 11. Herein, the lower surface 143 of the first gasket 14 is a surface on the opposite side to a surface to which the first plunger 12 is attached and is also a surface facing the bottom surface 111 in the first syringe barrel 11. The bottom surface 111 in the first syringe barrel 11 is a surface of the deepest portion in the first syringe barrel 11 and is also a surface in which a communication hole between the internal space of the first syringe barrel 11 and the internal space of the first port 13 is provided in an example.

When a liquid is stored in the first syringe barrel 11 via the first port 13, air in the gap between the lower surface 143 of the first gasket 14 and the bottom surface 111 of the first syringe barrel 11 is stored in the first syringe barrel 11 together with the liquid. When the amount of air in the gap is large, the upper limit of the volume of liquid that can be stored in the first syringe barrel 11 may be lowered. In a case in which the first buoy 51 is deformable in accordance with the shape of the gap between the lower surface 143 of the first gasket 14 and the bottom surface 111 in the first syringe barrel 11, the amount of air stored in the first syringe barrel 11 together with the liquid may be reduced to be small.

Similarly to the first buoy 51, the second buoy 52 may have flexibility. For example, the second buoy 52 is sandwiched between a lower surface 243 of the second gasket 24 and the bottom surface of the second syringe barrel 21 in a state in which no liquid is stored in the second syringe barrel 21 and the second gasket 24 is pushed to the deepest portion in the second syringe barrel 21. In this case, as illustrated in FIGS. 3 to 5, the second buoy 52 may be deformed in accordance with the shape of a gap between the lower surface 243 of the second gasket 24 and the bottom surface 211 in the second syringe barrel 21. Herein, the lower surface 243 of the second gasket 24 is a surface on the opposite side to a surface to which the second plunger 22 is attached and is also a surface facing the bottom surface 211 in the second syringe barrel 21. The bottom surface 211 in the second syringe barrel 21 is a surface of the deepest portion in the second syringe barrel 21 and is also a surface in which a communication hole between the internal space of the second syringe barrel 21 and the internal space of the second port 23 is provided in an example.

In a case in which the second buoy 52 is deformable in accordance with the shape of the gap between the lower surface 243 of the second gasket 24 and the bottom surface 211 in the second syringe barrel 21, the amount of air stored in the second syringe barrel 21 together with the liquid may be reduced to be small.

The first buoy 51 may function as a cover that blocks the movement of the liquid from the first syringe barrel 11 into the second syringe barrel 21 when part of the liquid (for example, the light layer L1) in the first syringe barrel 11 is moved into the second syringe barrel 21. In this case, the first buoy 51 may include a first covering portion capable of covering at least part of the first gasket 14. The shape of the first buoy 51 mentioned above will be described with reference to FIG. 6. FIG. 6 is a diagram illustrating an example of the shape of the first buoy 51 capable of functioning as a cover configured to block the movement of the liquid.

As illustrated in FIG. 6, the bottom surface 111 in the first syringe barrel 11 may be formed in a tapered shape in which the inner diameter decreases toward the first port 13 side. The lower surface 143 of the first gasket 14 may have an apex portion 1431 protruding most into the internal space in the first syringe barrel 11 and a peripheral edge portion 1432 surrounding the apex portion 1431 and may be formed in a tapered shape in which the outer diameter decreases from the peripheral edge portion 1432 toward the apex portion 1431. For example, the lower surface 143 of the first gasket 14 may have a conical surface as illustrated in FIG. 6.

As illustrated in FIG. 6, in the case where the first buoy 51 has a disk shape with a conical surface in which the center portion protrudes relative to the peripheral edge portion, when an angle at the apex of the conical surface of an upper surface 514 of the first buoy 51 is θb, θb may be smaller than θg, which is an angle of the apex portion 1431 of the lower surface 143 of the first gasket 14.

According to this configuration, after the first centrifugation processing, the first buoy 51 gradually approaches the first gasket 14 while the liquid of the light layer L1 in the first syringe barrel 11 is being stored in the second syringe barrel 21. When the liquid of the light layer L1 is substantially stored in the second syringe barrel 21, the peripheral edge portion of the upper surface 514 of the first buoy 51 makes contact with the lower surface 143 of the first gasket 14, and the liquid of the heavy layer H1 is blocked by the first buoy 51 and is unlikely to move into the second syringe barrel 21. In this way, the first buoy 51 may function as a cover that blocks the movement of the liquid from the first syringe barrel 11 into the second syringe barrel 21. Therefore, by using the syringe system 100, the liquid of the light layer L1 in the first syringe barrel 11 may be selectively moved into the second syringe barrel 21.

For example, when the first buoy 51 has a shape as illustrated in FIG. 6, the entire first buoy 51 functions as the first covering portion. However, the present disclosure is not limited thereto, and a site (for example, a center portion) of the first buoy 51 corresponding to a channel provided in the first gasket 14 may function as the first covering portion.

Alternatively, the first buoy 51 may function as a cover that blocks the movement of the liquid from inside the first syringe barrel 11 to the outside when part of the liquid (for example, the heavy layer H1) in the first syringe barrel 11 is moved to outside the first syringe barrel 11 through the first port 13. In this case, the first buoy 51 is required to include the first covering portion for closing the first port 13.

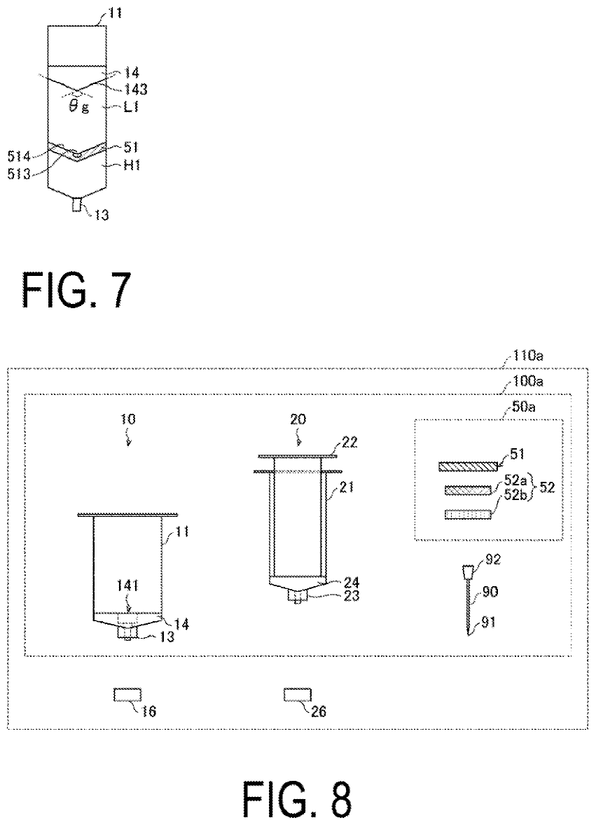

When the light layer L1 in the first syringe barrel 11 is moved into the second syringe barrel 21 by using the first needle 90, the first buoy 51 may function as a cover that blocks the movement of the liquid from the first syringe barrel 11 into the second syringe barrel 21. The shape of the first buoy 51 mentioned above will be described with reference to FIG. 7. FIG. 7 is a diagram illustrating an example of the shape of the first buoy 51 capable of functioning as a cover configured to block the movement of the liquid.

As illustrated in FIG. 7, in the case where the first buoy 51 has a disk shape with a conical surface in which the center portion protrudes relative to the peripheral edge portion, a recessed portion 513 may be provided in the upper surface 514 of the first buoy 51. An opening portion of the recessed portion 513 of the first buoy 51 is required to be in contact with the lower surface 143 of the first gasket 14.

According to this configuration, in the first centrifugation processing, the first buoy 51 gradually approaches the first gasket 14 while the liquid of the light layer L1 in the first syringe barrel 11 is being stored in the second syringe barrel 21. When the liquid of the light layer L1 is substantially stored in the second syringe barrel 21, the upper surface 514 of the first buoy 51 comes into contact with the lower surface 143 of the first gasket 14. At this time, since the recessed portion 513 covers the first end 91 of the first needle 90, the liquid of the heavy layer H1 is unlikely to move into the second syringe barrel 21. In this way, the first buoy 51 may function as a cover that blocks the movement of the liquid from the first syringe barrel 11 into the second syringe barrel 21. Therefore, by using the syringe system 100, the liquid of the light layer L1 in the first syringe barrel 11 may be selectively moved into the second syringe barrel 21. For example, when the first buoy 51 has a shape as illustrated in FIG. 7, the recessed portion 513 of the first buoy 51 functions as the first covering portion.

The second buoy 52 may function as a cover that blocks the movement of the liquid from the second syringe barrel 21 into a third syringe barrel 31 to be described later when part of the liquid (for example, the light layer L2) in the second syringe barrel 21 is moved into the third syringe barrel 31. In this case, the second buoy 52 may include a second covering portion capable of covering at least part of the second gasket 24. Similarly to the first buoy, the second buoy 52 may have a shape as illustrated in FIGS. 6 and 7. That is, the second buoy 52 may have a disk shape with a conical surface in which the center portion protrudes relative to the peripheral edge portion, or a recessed portion like the recessed portion 513 may be provided in the upper surface of the second buoy 52.

For example, when the second buoy 52 has a shape as illustrated in FIG. 6, the entire second buoy 52 functions as the second covering portion. However, the present disclosure is not limited thereto, and a site (for example, a center portion) of the second buoy 52 corresponding to a channel provided in the second gasket 24 may function as the second covering portion. For example, in a case where the second buoy 52 includes a recessed portion the same as or similar to the recessed portion 513 of the first buoy 51 illustrated in FIG. 7, the recessed portion of the second buoy 52 may function as the second covering portion.

Alternatively, the second buoy 52 may function as a cover that blocks the movement of the liquid from inside the second syringe barrel 21 to the outside when part of the liquid (for example, the heavy layer H2) in the second syringe barrel 21 is moved to outside the second syringe barrel 21 (see FIG. 16). In this case, the second buoy 52 is required to include the second covering portion for closing the second port 23.

First Variation

### Configuration of Syringe System 100a

The second buoy 52 may include two buoys having different masses per unit volume. FIG. 8 is a diagram illustrating a configuration example of a preparation kit 110a including a syringe system 100a equipped with the second buoy 52 including two buoys. The second buoy 52 of the syringe system 100a includes two buoys having different masses per unit volume. The syringe system 100a illustrated in FIG. 8 includes a buoy 52a having a specific gravity of Y and a buoy 52b having a specific gravity of Z different from the specific gravity of Y. Both the buoy 52a and the buoy 52b are positionable inside the second syringe barrel 21. In the case of performing the second centrifugation processing using centrifugal force directed from the second gasket 24 side toward the second port 23 side, the buoy 52a having a larger specific gravity than the buoy 52b may be located closer to the second port 23 relative to the buoy 52b.

For example, when a liquid in the second syringe barrel 21 is fractionated into the heavy layer H2 having a large specific gravity, the light layer L2 having a small specific gravity, and an intermediate layer M2 having an intermediate specific gravity between those of the heavy layer H2 and the light layer L2, the buoy 52a may be located at a boundary portion between the heavy layer H2 and the intermediate layer M2, and the buoy 52b may be located at a boundary portion between the intermediate layer M2 and the light layer L2 (see FIG. 18 and the like).

Second Variation

### Configuration of Syringe System 100b

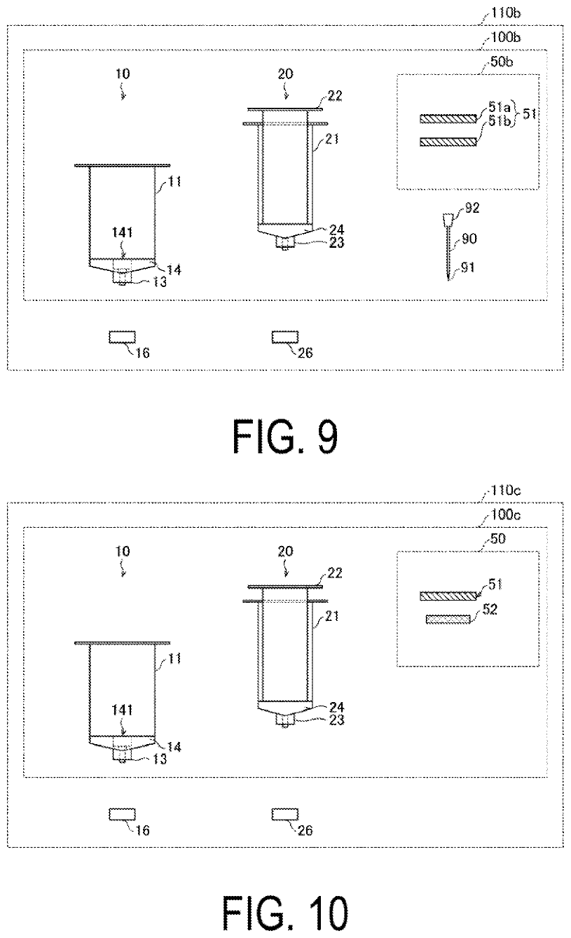

The first buoy 51 may include two buoys having different masses per unit volume. FIG. 9 is a diagram illustrating a configuration example of a preparation kit 110b including a syringe system 100b equipped with the first buoy 51 including two buoys. The first buoy 51 of the syringe system 100b includes a buoy 51a having a specific gravity of P and a buoy 51b having a specific gravity of Q different from the specific gravity of P. Both the buoy 51a and the buoy 51b are positionable inside the first syringe barrel 11. In the case of performing the first centrifugation processing using centrifugal force directed from the first gasket 14 side toward the first port 13 side, the buoy 51a having a larger specific gravity than the buoy 51b may be located closer to the first port 13 relative to the buoy 51b.

For example, when a liquid in the first syringe barrel 11 is fractionated into the heavy layer H1 having a large specific gravity, the light layer L1 having a small specific gravity, and the intermediate layer M1 having an intermediate specific gravity between those of the heavy layer H1 and the light layer L1, the buoy 51a may be located at the boundary portion between the heavy layer H1 and the intermediate layer M1, and the buoy 51b may be located at the boundary portion between the intermediate layer M1 and the light layer L1 (see FIG. 15 and the like).

For example, the buoy 51a may have a specific gravity between the density of red blood cells (about 1.102 g/ml) and the density of white blood cells (1.064 to 1.097 g/ml). For example, the density of the buoy 51a may be 1.07 to 1.10 g/ml, or may be 1.09 g/ml. The buoy 51a having such a density is located at the boundary portion between the heavy layer H1 and the intermediate layer M1 in the first syringe barrel 11 after the centrifugation processing. The buoy 51b may have a density between the density of platelets (1.03 to 1.04 g/ml) and the density of plasma (1.025 to 1.029 g/ml), for example. For example, the density of the buoy 51b may be 1.026 to 1.05 g/ml, or may be 1.03 g/ml. The buoy 51b having such density is located at the boundary portion between the intermediate layer M1 and the light layer L1 in the first syringe barrel 11 when the fractionation is carried out in the centrifugation processing (see FIG. 15).

The syringe system 100b illustrated in FIG. 9 is provided with only the first buoy 51 including two buoys having different specific gravities, but is not limited thereto. For example, the buoy 50b may further include the second buoy 52.

Third Variation

### Configuration of Syringe System 100c

The second syringe 20 may include the second port 23 (second leading end portion) capable of being coupled to the first gasket 14 and may be coupled to the first syringe 10 in a movable manner from the first syringe barrel 11. In this case, at least part of a liquid in the first syringe barrel 11 is movable into the second syringe barrel 21. This configuration makes it possible to move the liquid in the first syringe barrel 11 into the second syringe barrel 21 without using the first needle 90. FIG. 10 is a diagram illustrating a configuration example of a preparation kit 110c including a syringe system 100c, in which the second syringe 20 can be coupled to the first gasket 14. The syringe system 100c illustrated in FIG. 10 includes one first buoy 51 and one second buoy 52 but is not limited thereto. For example, the buoy 50 may include only any one of the first buoy 51 and the second buoy 52, or the first buoy 51 and the second buoy 52 may include a plurality of buoys.

First Gasket 14 and Second Port 23

The first attachment hole 141 to which the first plunger 12 can be attached is formed in the first gasket 14 of the first syringe 10 included in the syringe system 100c. The second port 23 included in the second syringe barrel 21 may also be attached to the first attachment hole 141. The structure of the first gasket 14 and second port 23 mentioned above will be described with reference to FIG. 11. FIG. 11 is a diagram illustrating an example of the structure adopted for coupling the first attachment hole 141 and the second port 23.

As illustrated in FIG. 11, a female screw 142 may be formed on the inner side surface of the first attachment hole 141 to be engaged with a male screw 232 formed on the outer side surface of the second port 23. As a result, the first gasket 14 and the second syringe barrel 21 may be coupled to each other in a liquid-tight manner.

The first attachment hole 141 need not penetrate inside and outside the first syringe barrel 11. Alternatively, as illustrated in FIG. 11, the first attachment hole 141 may penetrate inside and outside the first syringe barrel 11. Even in the case where the first attachment hole 141 penetrates inside and outside the first syringe barrel 11, the inside of the first syringe barrel 11 may be sealed when the second port 23 of the second syringe barrel 21, in which the second gasket 24 is inserted, is attached in the first attachment hole 141 and the first cap 16 is attached to the first port 13.

Second Syringe Barrel 21

The outer diameter of at least a part of the second syringe barrel 21 may be smaller than the inner diameter of the first syringe barrel 11. At least part of the second syringe 20 is positionable inside the first syringe barrel 11. The internal volume of the second syringe barrel 21 may be smaller than the internal volume of the first syringe barrel 11.

### Second Embodiment

Another embodiment of the present disclosure will be described below. For convenience of description, a member having the same function as that of a member described in the embodiments described above is denoted by the same reference sign, and description thereof will not be repeated.

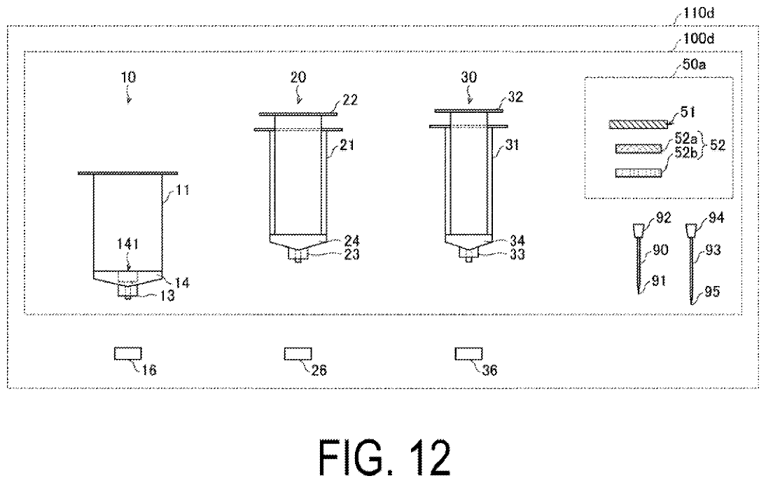

FIG. 12 is a diagram illustrating a configuration example of a preparation kit 110d including a syringe system 100d according to a second embodiment of the present disclosure. As illustrated in FIG. 12, the syringe system 100d further includes a third syringe 30 having the third syringe barrel 31 capable of holding a liquid therein. In FIG. 12, the syringe system 100d further including a second needle 93 is illustrated, but the second needle 93 is not an essential constituent element in the syringe system 100d.

Third Syringe 30

The third syringe 30 may include a third gasket 34 positionable inside the third syringe barrel 31. The third syringe 30 may include a third plunger 32 detachably provided to the third gasket 34.

The third syringe barrel 31 can hold the stored liquid. The third syringe barrel 31 constitutes at least part of the third syringe 30 along with the third gasket 34. A third port 33 (third leading end portion) is disposed at one end portion of the third syringe barrel 31. A portion of the third syringe barrel 31 for holding the liquid may have a substantially tubular shape.

The liquid held in the third syringe barrel 31 may be part of the liquid in the second syringe barrel 21 having been separated by the second centrifugation processing. As an example, the liquid held in the third syringe barrel 31 may be PRP, PPP, or the like.

As illustrated in FIG. 12, the preparation kit 110d may include a third cap 36 capable of being attached to the third port 33 in a liquid-tight manner. The third cap 36 is attached to the third port 33, for example, when the third syringe barrel 31 is used as a centrifugation container.

When the third cap 36 is attached to the third port 33, the third syringe barrel 31 may also function as a centrifugation container. The use of the third cap 36 makes it possible to perform centrifugation processing (third centrifugation processing to be described below) on a liquid in a state in which the liquid is stored in the third syringe barrel 31.

Second Needle 93

The second needle 93 is a tubular needle having a third end 95 capable of penetrating the second gasket 24 positionable inside the second syringe barrel 21 and a fourth end 94 located on the opposite side to the third end 95. The second needle 93 may be coupled to the third port 33 of the third syringe barrel 31. When the fourth end 94 of the second needle 93 is coupled to the third port 33 and the third end 95 of the second needle 93 penetrates the second gasket 24 located inside the second syringe barrel 21, the third syringe 30 may be coupled to the inside of the second syringe barrel 21 via the second needle 93.

The preparation kit 110d may further include a second needle guide 97 used when the third end 95 of the second needle 93 penetrates the second gasket 24. The second needle guide 97 includes a second guide hole serving as a through hole through which the third end 95 of the second needle 93 can move toward the second gasket 24. The third end 95 of the second needle 93 moves through the second guide hole and reaches the second gasket 24. The third end 95 having passed through the second guide hole penetrates the second gasket 24 and reaches the internal space in the second syringe barrel 21 (see FIG. 18 and the like).

The third syringe 30 may be coupled to the second syringe 20 in such a manner as to be movable from inside the second syringe barrel 21. In this case, in the second syringe 20 and the third syringe 30, the liquid may move from the second syringe barrel 21 into the third syringe barrel 31. The second needle 93 may be used when part of the liquid (for example, the light layer L2 or intermediate layer M2) in the second syringe barrel 21 is moved into the third syringe barrel 31.

Fourth Variation

### Configuration of Syringe System 100e

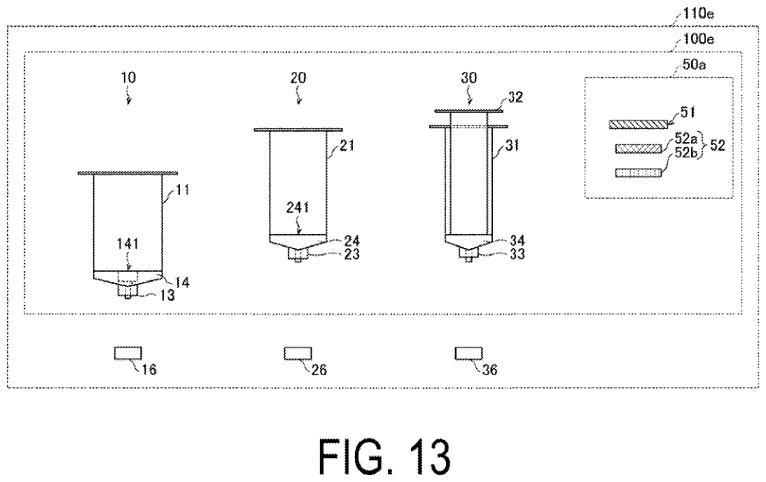

The third syringe 30 may have the third port 33 (third leading end portion) capable of being coupled to the second gasket 24. This configuration makes it possible to move the liquid in the second syringe barrel 21 into the third syringe barrel 31 without using the second needle 93. FIG. 13 is a diagram illustrating a configuration example of a preparation kit 110e including a syringe system 100e, in which the third syringe 30 can be coupled to the second gasket 24. The syringe system 100e illustrated in FIG. 13 is provided with the second buoy 52 including two buoys having different specific gravities but is not limited thereto. For example, a buoy 50a may include only one of the first buoy 51 or the second buoy 52, or may include one first buoy 51 and one second buoy 52.

Second Gasket 24 and Third Port 33

In the second gasket 24 of the second syringe 20 included in the syringe system 100e, a second attachment hole 241 is formed, in which the second plunger 22 can be attached. The third port 33 included in the third syringe barrel 31 may also be attached to the second attachment hole 241. The structure adopted for coupling the second attachment hole 241 and the third port 33 may be the same as that illustrated in FIG. 11, for example.

The second attachment hole 241 need not penetrate inside and outside the second syringe barrel 21. Alternatively, the second attachment hole 241 may penetrate inside and outside the second syringe barrel 21 similarly to the first attachment hole 141 (see FIG. 11). Even in the case where the second attachment hole 241 penetrates inside and outside the second syringe barrel 21, the inside of the second syringe barrel 21 may be sealed when the second plunger 22, the third port 33, or the like is attached in the second attachment hole 241, and the second cap 26 is attached to the second port 23.

Third Syringe Barrel 31

The outer diameter of at least a part of the third syringe barrel 31 may be smaller than the inner diameter of the second syringe barrel 21. At least part of the third syringe 30 is positionable inside the second syringe barrel 21. The internal volume of the third syringe barrel 31 may be smaller than the internal volume of the second syringe barrel 21.

Fifth Variation

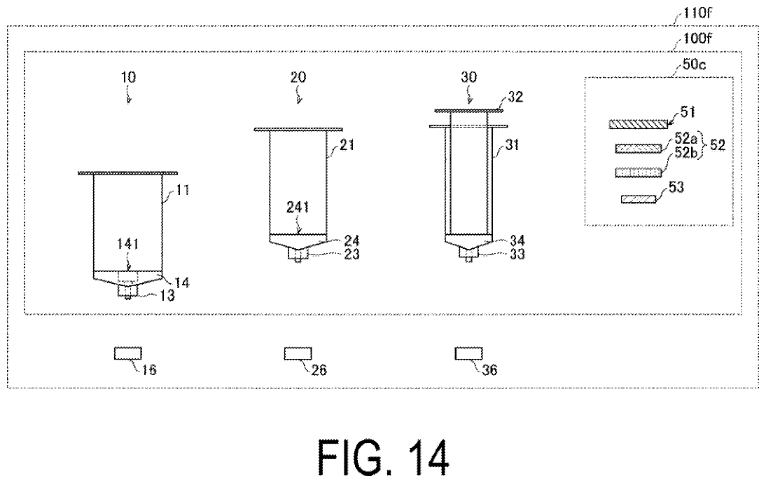

When the third syringe 30 is provided, the buoy 50 may include at least one third buoy 53 located inside the third syringe barrel 31. The third buoy is a buoy for the liquid held in the third syringe barrel 31. FIG. 14 is a diagram illustrating a configuration example of a preparation kit 110f including a syringe system 100f having the third buoy 53. A buoy 50c of the syringe system 100f further includes the third buoy 53 in addition to the first buoy 51 and the second buoy 52. The second buoy 52 includes the buoy 52a having a specific gravity of Y and the buoy 52b having a specific gravity of Z different from the specific gravity of Y. The third buoy 53 is positionable inside the third syringe barrel 31.

Third Buoy 53

The third buoy 53 is a buoy for the liquid in the third syringe barrel 31. That is, the third buoy 53 is a buoy located at a boundary portion between two or more layers when the liquid in the third syringe barrel 31 is fractionated into two or more layers. In other words, the third buoy 53 may have a specific gravity smaller than the specific gravity of a heavy layer H3 obtained by fractionating the liquid in the third syringe barrel 31, and larger than the specific gravity of a light layer L3 obtained by fractionating the liquid in the third syringe barrel 31. The third buoy 53 may include two or more buoy members having different specific gravities. The third buoy 53 may include, but is not limited to, at least any of buoy members made of polystyrene, polypropylene, polyethylene, silicone, acrylic resin, acrylonitrile-butadiene-styrene resin (ABS), acrylonitrile-styrene resin (AS), and combinations thereof.

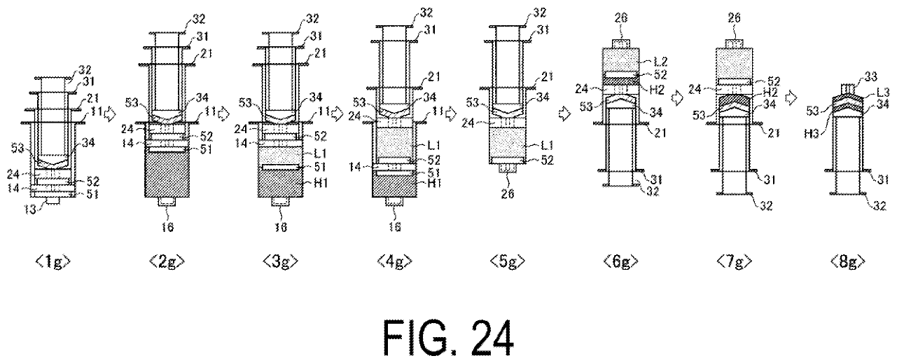

For example, when the liquid in the third syringe barrel 31 is fractionated into the heavy layer H3 having a large specific gravity and the light layer L3 having a small specific gravity, the third buoy 53 may be located at a boundary portion between the heavy layer H3 and the light layer L3 (see FIG. 24 and the like).

In the case of preparing PRP, the third centrifugation processing may be performed. By performing the third centrifugation processing, for example, PRP with a small mixed amount of white blood cells may be prepared from whole blood. Third centrifugation processing: the heavy layer H2 (the layer containing PRP) obtained by the second centrifugation processing is fractionated into the heavy layer H3 and the light layer L3 by using centrifugal force, and then the heavy layer H3 or the light layer L3 is separated by being taken out from the third syringe barrel 31 (see FIG. 24 and the like). The heavy layer H3 may be a layer having a higher concentration of white blood cells than the light layer L3.

The mass per unit volume of the third buoy 53 may be equal to or less than the mass per unit volume of the first buoy 51. The mass per unit volume of the third buoy 53 may be equal to or less than the mass per unit volume of the second buoy 52 (for example, the buoy 52a). The use of the third buoy 53 makes it possible to discharge any one of the liquid of the heavy layer H3 and the liquid of the light layer L3 through the third port 33 while reducing the mixing of the liquid of the heavy layer H3 containing white blood cells and the liquid of the light layer L3 containing platelets.

The third buoy 53 may have any of a through hole, a net-like portion, and a nonwoven fabric-like portion in at least one location. This configuration may cause the third buoy 53 not to obstruct the movement of each component of the liquid in the third syringe barrel 31 in the third centrifugation processing.

The third buoy 53 may have a substantially circular flat plate shape, a disk shape with a conical surface in which the center portion protrudes relative to the peripheral edge portion, or a disk shape with a curved surface in which the center portion protrudes relative to the peripheral edge portion. The diameter of the third buoy 53 may be smaller than the inner diameter of the third syringe barrel 31. As a result, the third buoy 53 is likely to move smoothly in the third syringe barrel 31. However, the present disclosure is not limited thereto as long as the third buoy 53 can move smoothly in the third syringe barrel 31.

The third buoy 53 has a first point P1 at any position on the lower surface and a second point P2 at any position on the upper surface. The length of at least one of the straight lines connecting the first point P1 and the second point P2 of the third buoy 53 may be larger than the inner diameter of the third syringe barrel 31. This makes it possible to suppress a changeover of the upper and lower surfaces of the third buoy 53 due to the rotation of the third buoy 53 in the third syringe barrel 31. In this case, the lower surface of the third buoy 53 is a surface facing the bottom surface of the third syringe barrel 31. The upper surface of the third buoy 53 is a surface on the opposite side to the lower surface of the third buoy 53.

Similarly to the first buoy 51 and the second buoy 52, the third buoy 53 may have flexibility.

The third buoy 53 may function as a cover that blocks the movement of the liquid from inside the third syringe barrel 31 to the outside when part of the liquid (for example, the light layer L3) in the third syringe barrel 31 is moved from the third port 33 to outside the third syringe barrel 31 through the third port 33. In this case, the third buoy 53 is required to include a third covering portion for closing the third port 33. A method for using the third buoy 53 discussed above will be described later (see FIG. 24).

### Example 1

PRP Preparation Method using Preparation Kit 110b
A process of preparing PRP using the preparation kit 110b will be described with reference to FIG. 15. FIG. 15 is a diagram illustrating a process flow for preparing PRP from blood by using the preparation kit 110b illustrated in FIG. 9.

State <1>

The first plunger 12 is attached to the first gasket 14 located inside the first syringe barrel 11. The first buoy 51 including the buoy 51a and the buoy 51b is located inside the first syringe barrel 11. The buoy 51b has a through hole while the buoy 51a does not have a through hole. In this case, a buoy having a specific gravity of P smaller than the specific gravity of red blood cells and larger than the specific gravity of white blood cells is used as the buoy 51a, and a buoy having a specific gravity of Q smaller than the specific gravity of platelets and larger than the specific gravity of plasma is used as the buoy 51b. The buoy 51b may include a net-like portion or a nonwoven fabric-like portion instead of the through hole.

State <2>

The first plunger 12 coupled to the first gasket 14 is pulled in a direction in which the first gasket 14 is pulled out of the first syringe barrel 11. As a result, the first gasket 14 moves, and the blood is stored in the first syringe barrel 11. After a predetermined amount of blood is stored in the first syringe barrel 11, the first cap 16 is attached to the first port 13.

State <3> Centrifugation Processing

After removing the first plunger 12 coupled to the first gasket 14 from the first syringe barrel 11, the first syringe barrel 11 is used as a centrifugation container, and centrifugation processing is performed using centrifugal force directed from the first gasket 14 side toward the first port 13 side. By this centrifugation processing, the liquid in the first syringe barrel 11 is fractionated into the heavy layer H1 containing red blood cells, the intermediate layer M1 containing PRP, and the light layer L1 (PPP layer). When the buoy 51a having a specific gravity smaller than the specific gravity of the heavy layer H1 and larger than the specific gravity of the intermediate layer M1 is used, the buoy 51a will be located at a boundary portion between the heavy layer H1 and the intermediate layer M1. When the buoy 51b having a specific gravity smaller than the specific gravity of the intermediate layer M1 and larger than the specific gravity of the light layer L1 is used, the buoy 51b will be located at a boundary portion between the intermediate layer M1 and the light layer L1.

State <4> Extraction Processing

After the first needle 90 is attached to the second syringe barrel 21, the first end 91 of the first needle 90 is made to penetrate the first gasket 14. At this time, the first needle guide 96 having the first guide hole into which the first end 91 of the first needle 90 can be inserted may be used. The first end 91 having passed through the first guide hole penetrates the first gasket 14, further penetrates the through hole of the buoy 51b, and consequently reaches a position where the liquid of the intermediate layer M1 in the first syringe barrel 11 can be sucked.

The second plunger 22 is pulled in a direction in which the second gasket 24 is pulled out of the second syringe barrel 21. As a result, the second gasket 24 moves, and the liquid of the intermediate layer M1 is stored in the second syringe barrel 21. The liquid of the heavy layer H1 mainly containing red blood cells and the light layer L1 (PPP layer) remain in the first syringe barrel 11, from which the liquid of the intermediate layer M1 has been taken out. The movement of the second gasket 24 in the direction in which the second syringe barrel 21 is pulled out may be ended when the buoy 51b comes into contact with the buoy 51a. Thus, the liquid of the intermediate layer M1 in the first syringe barrel 11 may be selectively moved into the second syringe barrel 21. When the buoy 51b comes into contact with the buoy 51a, the buoy 51a may close the channel of the liquid from the first needle 90 into the second syringe barrel 21. In this case, the liquid of the intermediate layer M1 in the first syringe barrel 11 may be more selectively moved into the second syringe barrel 21. That is, the possibility of the movement of the liquid of the heavy layer H1 (and the liquid of the light layer L1) in the first syringe barrel 11 into the second syringe barrel 21 may be lowered.

State <5>

As described above, when the preparation kit 110b is used, the liquid of the intermediate layer M1 containing PRP may be stored in the second syringe barrel 21 by performing centrifugation processing once. For example, when an injection needle (not illustrated) different from the first needle 90 is attached to the second port 23, the PRP may be administered to an affected area of the patient by using the second syringe 20.

### Example 2

PRP Preparation Method 1 using Preparation Kit 110c
A process of preparing PRP using the preparation kit 110c will be described with reference to FIG. 16. FIG. 16 is a diagram illustrating an example of a process flow for preparing PRP from blood by using the preparation kit 110c illustrated in FIG. 10.

State <1a>

The second port 23 of the second syringe barrel 21 is screwed into the first gasket 14 of the first syringe barrel 11. The second plunger 22 is attached to the second gasket 24 of the second syringe barrel 21. Thus, a syringe unit in which the first syringe barrel 11 and the second syringe barrel 21 are coupled to each other is formed.

The first buoy 51 is located inside the first syringe barrel 11, and the second buoy 52 is located inside the second syringe barrel 21.

State <2a>

The second syringe 20 coupled to the first gasket 14 is pulled in a direction in which the first gasket 14 is pulled out of the first syringe barrel 11. As a result, the first gasket 14 moves, and the blood is stored in the first syringe barrel 11. At this time, the second syringe 20 serves as a plunger for moving the first gasket 14.

After a predetermined amount of blood is stored in the first syringe barrel 11, the first cap 16 is attached to the first port 13.

State <3a> First Centrifugation Processing

After the first cap 16 is attached to the first port 13, the first syringe barrel 11 is used as a centrifugation container to perform the first centrifugation processing using centrifugal force directed from the first gasket 14 side toward the first port 13 side, whereby the blood is fractionated into the heavy layer H1 mainly containing red blood cells and the light layer L1 containing white blood cells and platelets. When the first buoy 51 having a specific gravity smaller than the specific gravity of the heavy layer H1 and larger than the specific gravity of the light layer L1 is used, the first buoy 51 is located at a boundary portion between the heavy layer H1 and the light layer L1.

State <4a> Extraction Processing

The second plunger 22 is pulled in a direction in which the second gasket 24 is pulled out of the second syringe barrel 21. As a result, the second gasket 24 moves, and the liquid of the light layer L1 is stored in the second syringe barrel 21. The movement of the second gasket 24 in the direction in which the second syringe barrel 21 is pulled out may be ended when the first buoy 51 comes into contact with the first gasket 14. Thus, the liquid of the light layer L1 in the first syringe barrel 11 may be selectively moved into the second syringe barrel 21. When the first buoy 51 comes into contact with the first gasket 14, the first buoy 51 may close the liquid channel (for example, the first attachment hole 141) provided in the first gasket 14 into the second syringe barrel 21. In this case, the liquid of the light layer L1 in the first syringe barrel 11 may be more selectively moved into the second syringe barrel 21. That is, the possibility of the movement of the liquid of the heavy layer H1 in the first syringe barrel 11 into the second syringe barrel 21 may be lowered.

State <5a>

After the second syringe 20 is removed from the first gasket 14, the second cap 26 is attached to the second port 23.

State <6a> Second Centrifugation Processing

After the second cap 26 is attached to the second port 23, the second syringe barrel 21 is used as a centrifugation container to perform the second centrifugation processing using centrifugal force directed from the second gasket 24 side toward the second port 23 side. By the second centrifugation processing, the liquid in the second syringe barrel 21 is fractionated into the heavy layer H2 containing PRP and the light layer L2 (PPP layer). When the second buoy 52 having a specific gravity smaller than the specific gravity of the heavy layer H2 and larger than the specific gravity of the light layer L2 is used, the second buoy 52 is located at a boundary portion between the heavy layer H2 and the light layer L2.

State <7a>

When the second cap 26 is removed from the second port 23 and an optional injection needle 99 is attached instead, the heavy layer H2 containing PRP may be administered to an affected area of the patient from inside the second syringe barrel 21. In this case, the second plunger 22 is pushed in a direction in which the second gasket 24 is pushed into the second syringe barrel 21. As a result, the second gasket 24 moves, and the liquid of the heavy layer H2 moves from inside the second syringe barrel 21 toward the affected area of the patient. The movement of the second gasket 24 in the pushing direction into the second syringe barrel 21 may be ended when the first buoy 51 comes into contact with the bottom surface in the second syringe barrel 21 (see FIG. 3 to FIG. 5). Thus, the liquid of the heavy layer H2 in the second syringe barrel 21 may be selectively moved into the affected area of the patient. When the second buoy 52 comes into contact with the bottom surface 211 of the second syringe barrel 21, the second buoy 52 may close the second port 23. In this case, the liquid of the heavy layer H2 in the second syringe barrel 21 may be more selectively moved into the affected area of the patient. That is, the possibility of the movement of the liquid of the light layer L2 in the second syringe barrel 21 into the affected area of the patient may be lowered.

### Example 3

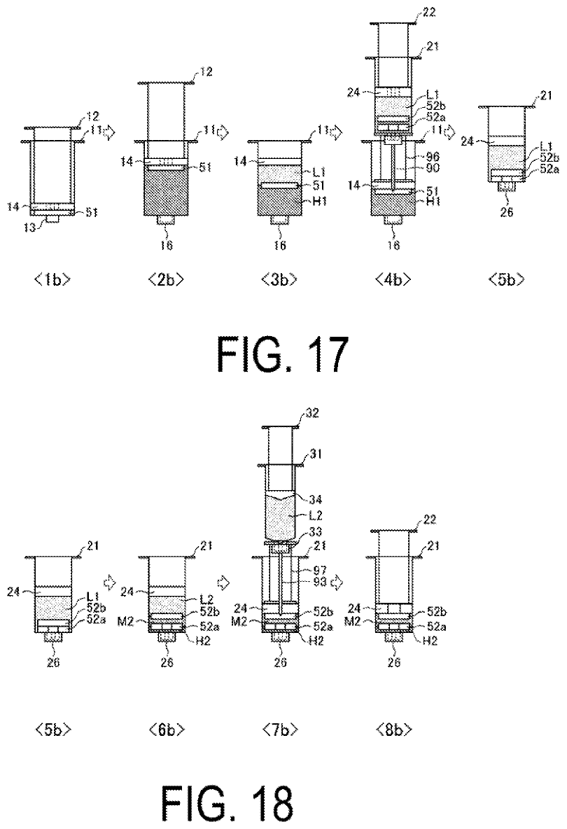

PRP Preparation Method 1 using Preparation Kit 110d
A process of preparing PRP using the preparation kit 110d will be described with reference to FIGS. 17 and 18. FIG. 17 is a diagram illustrating an example of the first half of a process flow for preparing PRP from blood by using the preparation kit 110d illustrated in FIG. 12. FIG. 18 is a diagram illustrating an example of the second half of the process flow for preparing PRP from blood by using the preparation kit 110d illustrated in FIG. 12.

State <1b>

The first plunger 12 is attached to the first gasket 14 located inside the first syringe barrel 11. At this time, the first buoy 51 is located inside the first syringe barrel 11.

State <2b>

The first plunger 12 coupled to the first gasket 14 is pulled in a direction in which the first gasket 14 is pulled out of the first syringe barrel 11. As a result, the first gasket 14 moves, and the blood is stored in the first syringe barrel 11. After a predetermined amount of blood is stored in the first syringe barrel 11, the first cap 16 is attached to the first port 13. The first plunger 12 is removed from the first gasket 14.

State <3b> First Centrifugation Processing

The first syringe barrel 11 is used as a centrifugation container to perform the first centrifugation processing using centrifugal force directed from the first gasket 14 side toward the first port 13 side. By the first centrifugation processing, for example, the blood is fractionated into the heavy layer H1 mainly containing red blood cells and the light layer L1 containing white blood cells and platelets. When the first buoy 51 having a specific gravity smaller than the specific gravity of the heavy layer H1 and larger than the specific gravity of the light layer L1 is used, the first buoy 51 is located at a boundary portion between the heavy layer H1 and the light layer L1.

State <4b> Extraction Processing

The second buoy 52 including the buoy 52a and the buoy 52b is located inside the second syringe barrel 21. The buoy 52a has a through hole while the buoy 52b does not have a through hole. In this case, a buoy having a specific gravity of Y smaller than the specific gravity of white blood cells such as neutrophils and basophils and larger than the specific gravity of platelets is used as the buoy 52a, and a buoy having a specific gravity of Z smaller than the specific gravity of platelets and larger than the specific gravity of plasma is used as the buoy 52b. The buoy 52a may include a net-like portion or a nonwoven fabric-like portion instead of the through hole.

After the first needle 90 is attached to the second syringe barrel 21, the first end 91 of the first needle 90 is made to penetrate the first gasket 14. At this time, the first needle guide 96 having the first guide hole into which the first end 91 of the first needle 90 can be inserted may be used. The first end 91 having passed through the first guide hole penetrates the first gasket 14 to reach a position in the internal space in the first syringe barrel 11 where the liquid of the light layer L1 in the first syringe barrel 11 can be sucked.

The second plunger 22 is pulled in a direction in which the second gasket 24 is pulled out of the second syringe barrel 21. As a result, the second gasket 24 moves, and the liquid of the light layer L1 is stored in the second syringe barrel 21. The movement of the second gasket 24 in the direction in which the second syringe barrel 21 is pulled out may be ended when the first buoy 51 comes into contact with the first gasket 14. Thus, the liquid of the light layer L1 in the first syringe barrel 11 may be selectively moved into the second syringe barrel 21. When the first buoy 51 comes into contact with the first gasket 14, the first buoy 51 may close the channel of the liquid from the first needle 90 into the second syringe barrel 21. In this case, the liquid of the light layer L1 in the first syringe barrel 11 may be more selectively moved into the second syringe barrel 21. That is, the possibility of movement of the liquid of the heavy layer H1 in the first syringe barrel 11 into the second syringe barrel 21 may be lowered.

State <5b>

As a result, the liquid of the heavy layer H1 mainly containing red blood cells remains in the first syringe barrel 11, from which the liquid of the light layer L1 has been taken out, and the liquid of the light layer L1 having been fractionated by the first centrifugation processing is stored in the second syringe barrel 21. The second plunger 22 is removed from the second gasket 24. Here, the first half of the process flow for preparing PRP from blood ends.

The second half of the process flow for preparing PRP from blood will be described with reference to FIG. 18. The state <5b> illustrated in FIG. 18 is the same as the state <5b> illustrated in FIG. 17.

State <6b> Second Centrifugation Processing

The second syringe barrel 21 is used as a centrifugation container to perform the second centrifugation processing using centrifugal force directed from the second gasket 24 side toward the second port 23 side. By the second centrifugation processing, the liquid in the second syringe barrel 21 is fractionated into the heavy layer H2 containing white blood cells, the intermediate layer M2 containing PRP, and the light layer L2 (PPP layer). When the buoy 52a having a specific gravity smaller than the specific gravity of the heavy layer H2 and larger than the specific gravity of the intermediate layer M2 is used, the buoy 52a is located at a boundary portion between the heavy layer H2 and the intermediate layer M2. When the buoy 52b having a specific gravity smaller than the specific gravity of the intermediate layer M2 and larger than the specific gravity of the light layer L2 is used, the buoy 52b is located at a boundary portion between the intermediate layer M2 and the light layer L2.

State <7b> Extraction Processing

After the second needle 93 is attached to the third syringe barrel 31, the third end 95 of the second needle 93 is made to penetrate the second gasket 24. At this time, the second needle guide 97 having the second guide hole into which the third end 95 of the second needle 93 can be inserted may be used. The third end 95 having passed through the second guide hole penetrates the second gasket 24 to reach a position where the liquid of the light layer L2 in the second syringe barrel 21 can be sucked.

The third plunger 32 is pulled in a direction in which the third gasket 34 is pulled out of the third syringe barrel 31. As a result, the third gasket 34 moves, and the liquid of the light layer L2 is stored in the third syringe barrel 31. The movement of the third gasket 34 in the direction in which the third syringe barrel 31 is pulled out may be ended when the buoy 52b comes into contact with the second gasket 24. Thus, the liquid of the light layer L2 in the second syringe barrel 21 may be selectively moved into the third syringe barrel 31. When the buoy 52b comes into contact with the second gasket 24, the buoy 52b may close the channel of the liquid from the second needle 93 into the second syringe barrel 21. In this case, the liquid of the light layer L1 in the second syringe barrel 21 may be more selectively moved into the third syringe barrel 31. That is, the possibility of movement of at least any of the liquid of the heavy layer H2 and the intermediate layer M2 in the second syringe barrel 21 into the third syringe barrel 31 may be lowered.

State <8b>

As a result, the heavy layer H2 containing white blood cells at a high concentration and the intermediate layer M2 containing PRP with a low concentration of white blood cells remain in the second syringe barrel 21, from which the liquid of the light layer L2 has been taken out. For example, when the second plunger 22 is reattached to the second gasket 24 and the second cap 26 is removed from the second port 23, only the liquid of the layer closer to the second port 23 relative to the buoy 52a (that is, the heavy layer H2) may be discharged from the second port 23.

The buoy 52a has a through hole as illustrated in FIG. 18. With this, the liquid of the intermediate layer M2 may also be discharged from the second port 23. In this case, an injection needle (not illustrated) may be attached to the second port 23 to administer PRP with a low concentration of white blood cells into an affected area of the patient.

### Example 4

### PRP Preparation Method 2 Using Preparation Kit 110d

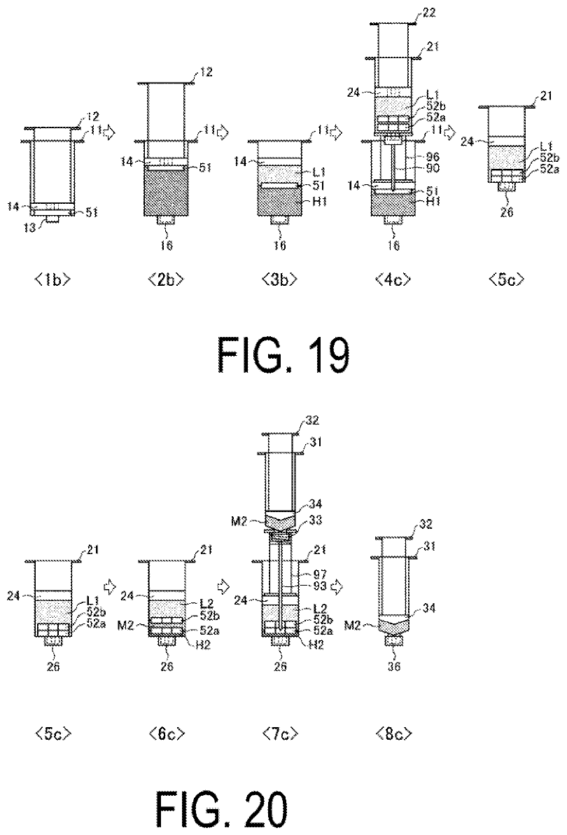

Both of the buoy 52a and the buoy 52b in the second syringe barrel 21 may have a through hole. A process of preparing PRP using the preparation kit 110d will be described with reference to FIGS. 19 and 20. FIG. 19 is a diagram illustrating an example of the first half of a process flow for preparing PRP from blood by using the preparation kit 110d illustrated in FIG. 12. FIG. 20 is a diagram illustrating an example of the second half of the process flow for preparing PRP from blood by using the preparation kit 110d illustrated in FIG. 12. For convenience of description, a state identical to the state described in the above examples is denoted by the same reference sign, and description thereof will not be repeated.

State <4c>

The second buoy 52 including the buoy 52a and the buoy 52b is located inside the second syringe barrel 21. The buoy 52a and the buoy 52b each have a through hole. In this case, a buoy having a specific gravity of Y smaller than the specific gravity of white blood cells such as neutrophils and basophils and larger than the specific gravity of platelets is used as the buoy 52a, and a buoy having a specific gravity of Z smaller than the specific gravity of platelets and larger than the specific gravity of plasma is used as the buoy 52b. The buoy 52a and the buoy 52b may each include a net-like portion or a nonwoven fabric-like portion instead of the through hole.

State <5c>

The liquid of the heavy layer H1 mainly containing red blood cells remains in the first syringe barrel 11, from which the liquid of the light layer L1 has been taken out, and the liquid of the light layer L1 having been fractionated by the first centrifugation processing is stored in the second syringe barrel 21. The second plunger 22 is removed from the second gasket 24. Here, the first half of the process flow for preparing PRP from blood ends.

The second half of the process flow for preparing PRP from blood will be described with reference to FIG. 20. The state <5c> illustrated in FIG. 20 is the same as the state <5c> illustrated in FIG. 19.

State <6c> Second Centrifugation Processing

The second syringe barrel 21 is used as a centrifugation container to perform the second centrifugation processing using centrifugal force directed from the second gasket 24 side toward the second port 23 side. By the second centrifugation processing, the liquid in the second syringe barrel 21 is fractionated into the heavy layer H2 containing white blood cells, the intermediate layer M2 containing PRP, and the light layer L2 (PPP layer). When the buoy 52a having a specific gravity smaller than the specific gravity of the heavy layer H2 and larger than the specific gravity of the intermediate layer M2 is used, the buoy 52a is located at a boundary portion between the heavy layer H2 and the intermediate layer M2. When the buoy 52b having a specific gravity smaller than the specific gravity of the intermediate layer M2 and larger than the specific gravity of the light layer L2 is used, the buoy 52b is located at a boundary portion between the intermediate layer M2 and the light layer L2.

State <7c> Extraction Processing

After the second needle 93 is attached to the third syringe barrel 31, the third end 95 of the second needle 93 is made to penetrate the second gasket 24. At this time, the second needle guide 97 having the second guide hole into which the third end 95 of the second needle 93 can be inserted may be used. The third end 95 having passed through the second guide hole penetrates the second gasket 24, further penetrates the through hole of the buoy 52b, and consequently reaches a position where the liquid of the intermediate layer M2 in the second syringe barrel 21 can be sucked.

The third plunger 32 is pulled in a direction in which the third gasket 34 is pulled out of the third syringe barrel 31. As a result, the third gasket 34 moves, and the liquid of the intermediate layer M2 is stored in the third syringe barrel 31. The movement of the third gasket 34 in the direction in which the third syringe barrel 31 is pulled out may be ended when the buoy 52b comes into contact with the buoy 52a. Thus, the liquid of the intermediate layer M2 in the second syringe barrel 21 may be selectively moved into the third syringe barrel 31.

State <8c>

As a result, the intermediate layer M2 containing PRP is stored in the third syringe barrel 31. An injection needle (not illustrated) may be attached to the third port 33 to administer the PRP into an affected area of the patient.

### Example 5

PRP Preparation Method 1 using Preparation Kit 110e

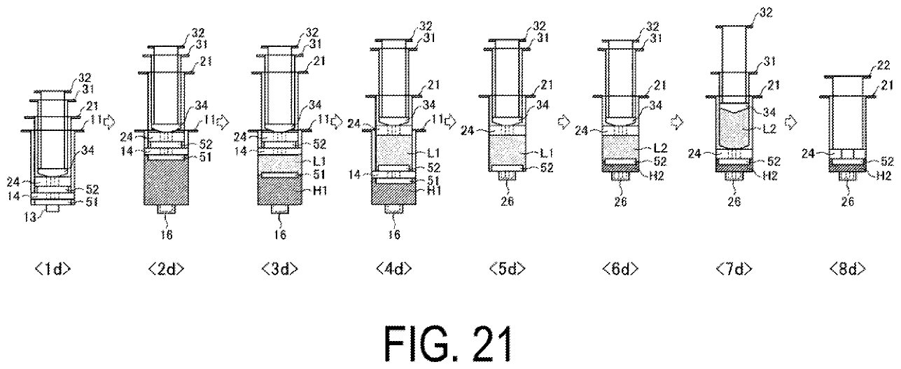

A process of preparing PRP using a preparation kit including one first buoy 51 and one second buoy 52 will be described with reference to FIG. 21. FIG. 21 is a diagram illustrating an example of a process flow for preparing PRP from blood by using the preparation kit 110e including one first buoy 51 and one second buoy 52 illustrated in FIG. 13.

State <1d>

The second port 23 of the second syringe barrel 21 is screwed into the first gasket 14 of the first syringe barrel 11, and the third port 33 of the third syringe barrel 31 is screwed into the second gasket 24 of the second syringe barrel 21. Thus, a syringe unit is configured in which the first syringe barrel 11 and the second syringe barrel 21 are coupled, and further the second syringe barrel 21 and the third syringe barrel 31 are coupled.

The first buoy 51 is located inside the first syringe barrel 11, and the second buoy 52 is located inside the second syringe barrel 21.

State <2d>

A syringe unit in which the second syringe barrel 21 coupled to the first gasket 14 and the third syringe 30 coupled to the second gasket 24 are combined is pulled in a direction in which the first gasket 14 is pulled out of the first syringe barrel 11. As a result, the first gasket 14 moves, and the blood is stored in the first syringe barrel 11. At this time, the syringe unit combining the second syringe barrel 21 and the third syringe 30 serves as a plunger for moving the first gasket 14.

After a predetermined amount of blood is stored in the first syringe barrel 11, the first cap 16 is attached to the first port 13.

State <3d> First Centrifugation Processing

After the first cap 16 is attached to the first port 13, the first syringe barrel 11 is used as a centrifugation container to perform the first centrifugation processing using centrifugal force directed from the first gasket 14 side toward the first port 13 side, whereby the blood is fractionated into the heavy layer H1 mainly containing red blood cells and the light layer L1 containing white blood cells and platelets. When the first buoy 51 having a specific gravity smaller than the specific gravity of the heavy layer H1 and larger than the specific gravity of the light layer L1 is used, the first buoy 51 is located at a boundary portion between the heavy layer H1 and the light layer L1.

State <4d> Extraction Processing

The third syringe 30 coupled to the second gasket 24 is pulled in a direction in which the second gasket 24 is pulled out of the second syringe barrel 21. As a result, the second gasket 24 moves, and the liquid of the light layer L1 is stored in the second syringe barrel 21. The movement of the second gasket 24 in the direction in which the second syringe barrel 21 is pulled out may be ended when the first buoy 51 comes into contact with the second gasket 24. Thus, the liquid of the light layer L1 in the first syringe barrel 11 may be selectively moved into the second syringe barrel 21. When the first buoy 51 comes into contact with the first gasket 14, the first buoy 51 may close the liquid channel (for example, the first attachment hole 141) provided in the first gasket 14 into the second syringe barrel 21. In this case, the liquid of the light layer L1 in the first syringe barrel 11 may be more selectively moved into the second syringe barrel 21. That is, the possibility of movement of the liquid of the heavy layer H1 in the first syringe barrel 11 into the second syringe barrel 21 may be lowered.

State <5d>

After the syringe unit combining the second syringe barrel 21 and the third syringe 30 is removed from the first gasket 14, the second cap 26 is attached to the second port 23.

State <6d> Second Centrifugation Processing

After the second cap 26 is attached to the second port 23, the second syringe barrel 21 is used as a centrifugation container to perform the second centrifugation processing using centrifugal force directed from the second gasket 24 side toward the second port 23 side. By the second centrifugation processing, the liquid in the second syringe barrel 21 is fractionated into the heavy layer H2 containing PRP and the light layer L2 (PPP layer). When the second buoy 52 having a specific gravity smaller than the specific gravity of the heavy layer H2 and larger than the specific gravity of the light layer L2 is used, the second buoy 52 is located at a boundary portion between the heavy layer H2 and the light layer L2.

State <7d> Extraction Processing

The third plunger 32 coupled to the third gasket 34 is pulled in a direction in which the third gasket 34 is pulled out of the third syringe barrel 31. As a result, the third gasket 34 moves, and the liquid of the light layer L2 is stored in the third syringe barrel 31. The movement of the third gasket 34 in the direction in which the third syringe barrel 31 is pulled out may be ended when the second buoy 52 comes into contact with the second gasket 24. Thus, the liquid of the light layer L2 in the second syringe barrel 21 may be selectively moved into the third syringe barrel 31. When the second buoy 52 comes into contact with the second gasket 24, the second buoy 52 may close the liquid channel (for example, the second attachment hole 241) provided in the second gasket 24 into the third syringe barrel 31. In this case, the liquid of the light layer L2 in the second syringe barrel 21 may be more selectively moved into the third syringe barrel 31. That is, the possibility of movement of the liquid of the heavy layer H2 in the second syringe barrel 21 into the third syringe barrel 31 may be lowered. As a result, only the heavy layer H2 containing PRP remains in the second syringe barrel 21.

State <8d>

The third syringe 30 is detached from the second gasket 24, and the second plunger 22 is attached to the second gasket 24 instead. With this, the liquid containing PRP in the second syringe barrel 21 may be discharged from the second syringe barrel 21 by using the second plunger 22. An injection needle (not illustrated) may be attached to the second port 23 to administer the PRP into an affected area of the patient.

### Example 6

PRP Preparation Method 2 using Preparation Kit 110e

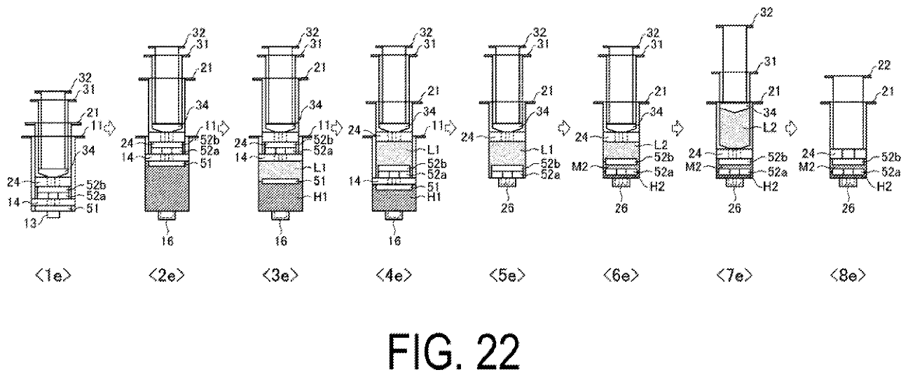

A process of preparing PRP using the preparation kit 110e including the buoy 52a and buoy 52b as the second buoy 52 will be described with reference to FIG. 22. FIG. 22 is a diagram illustrating another example of a process flow for preparing PRP from blood by using the preparation kit 110e illustrated in FIG. 13.

State <1e>

The second port 23 of the second syringe barrel 21 is screwed into the first gasket 14 of the first syringe barrel 11, and the third port 33 of the third syringe barrel 31 is screwed into the second gasket 24 of the second syringe barrel 21. Thus, a syringe unit is configured in which the first syringe barrel 11 and the second syringe barrel 21 are coupled, and further the second syringe barrel 21 and the third syringe barrel 31 are coupled. The buoy 52a and the buoy 52b are located inside the second syringe barrel 21.

The first buoy 51 is located in the first syringe barrel 11, and the buoy 52a and buoy 52b are located inside the second syringe barrel 21.

State <2e> to State <5e>

Each of the states <2e> to <5e> indicates a state the same as or similar to each of the above-described states <2d> to <5d> except that the buoy 52a and buoy 52b are located inside the second syringe barrel 21.

State <6e> Second Centrifugation Processing

After the second cap 26 is attached to the second port 23, the second syringe barrel 21 is used as a centrifugation container to perform the second centrifugation processing using centrifugal force directed from the second gasket 24 side toward the second port 23 side. By the second centrifugation processing, the liquid in the second syringe barrel 21 is fractionated into the heavy layer H2 containing white blood cells, the intermediate layer M2 containing PRP, and the light layer L2 (PPP layer). When the buoy 52a having a specific gravity smaller than the specific gravity of the heavy layer H2 and larger than the specific gravity of the intermediate layer M2 is used, the buoy 52a is located at a boundary portion between the heavy layer H2 and the intermediate layer M2. When the buoy 52b having a specific gravity smaller than the specific gravity of the intermediate layer M2 and larger than the specific gravity of the light layer L2 is used, the buoy 52b is located at a boundary portion between the intermediate layer M2 and the light layer L2.

State <7e> Extraction Processing

The third plunger 32 coupled to the third gasket 34 is pulled in a direction in which the third gasket 34 is pulled out of the third syringe barrel 31. As a result, the third gasket 34 moves, and the liquid of the light layer L2 is stored in the third syringe barrel 31. The movement of the third gasket 34 in the direction in which the third syringe barrel 31 is pulled out may be ended when the buoy 52b comes into contact with the second gasket 24. Thus, the liquid of the light layer L2 in the second syringe barrel 21 may be selectively moved into the third syringe barrel 31. When the buoy 52b comes into contact with the second gasket 24, the buoy 52b may close the liquid channel (for example, the second attachment hole 241) provided in the second gasket 24 into the third syringe barrel 31. In this case, the liquid of the light layer L2 in the second syringe barrel 21 may be more selectively moved into the third syringe barrel 31. That is, the possibility of movement of the liquid of the intermediate layer M2 and the liquid of the heavy layer H2 in the second syringe barrel 21 into the third syringe barrel 31 may be lowered.

State <8e>

As a result, the heavy layer H2 containing white blood cells and the intermediate layer M2 containing PRP remain in the second syringe barrel 21, from which the liquid of the light layer L2 has been taken out. For example, when the second plunger 22 is attached to the second gasket 24 and the second cap 26 is removed from the second port 23, only the liquid of the layer closer to the second port 23 relative to the buoy 52a (that is, the heavy layer H2) may be discharged from the second port 23. With this, the amount of white blood cells contained in PRP may be reduced.

The buoy 52a has a through hole as illustrated in FIG. 22. With this, the liquid of the intermediate layer M2 may also be discharged from the second port 23. In this case, an injection needle (not illustrated) may be attached to the second port 23 to administer the PRP into an affected area of the patient.

### Example 7

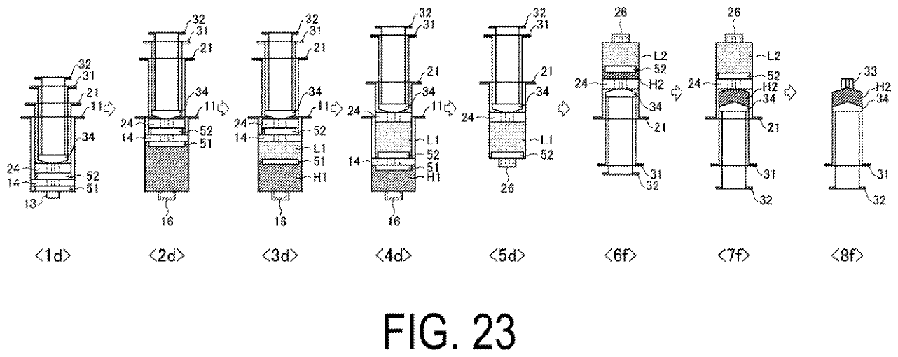

PRP Preparation Method 3 using Preparation Kit 110e
A process of preparing PRP using the preparation kit 110e will be described with reference to FIG. 23. FIG. 23 is a diagram illustrating another example of a process flow for preparing PRP from blood by using the preparation kit 110e illustrated in FIG. 13. For convenience of description, a state identical to the state described in the above examples is denoted by the same reference sign, and description thereof will not be repeated.

State <6f Second Centrifugation Processing

After the second cap 26 is attached to the second port 23, the second syringe barrel 21 is used as a centrifugation container to perform the second centrifugation processing using centrifugal force directed from the second port 23 side toward the second gasket 24 side. By the second centrifugation processing, the liquid in the second syringe barrel 21 is fractionated into the heavy layer H2 containing PRP and the light layer L2 (PPP layer). When the second buoy 52 having a specific gravity smaller than the specific gravity of the heavy layer H2 and larger than the specific gravity of the light layer L2 is used, the second buoy 52 is located at a boundary portion between the heavy layer H2 and the light layer L2. Unlike the state <6c> illustrated in FIG. 20, the heavy layer H2 is located on the second gasket 24 side, and the light layer L2 is located on the second port 23 side.

State <7f Extraction Processing

The third plunger 32 coupled to the third gasket 34 is pulled in a direction in which the third gasket 34 is pulled out of the third syringe barrel 31. As a result, the third gasket 34 moves, and the liquid of the heavy layer H2 is stored in the third syringe barrel 31. The movement of the third gasket 34 in the direction in which the third syringe barrel 31 is pulled out may be ended when the second buoy 52 comes into contact with the second gasket 24. Thus, the liquid of the heavy layer H2 in the second syringe barrel 21 may be selectively moved into the third syringe barrel 31. When the second buoy 52 comes into contact with the second gasket 24, the second buoy 52 may close the liquid channel (for example, the second attachment hole 241) provided in the second gasket 24 into the third syringe barrel 31. In this case, the liquid of the heavy layer H2 in the second syringe barrel 21 may be more selectively moved into the third syringe barrel 31. That is, the possibility of movement of the liquid of the light layer L2 in the second syringe barrel 21 into the third syringe barrel 31 may be lowered.

State <8f>

The third syringe 30 is removed from the second gasket 24. With this, the heavy layer H2 as a liquid containing PRP in the third syringe barrel 31 may be discharged from the third syringe barrel 31 by using the third plunger 32. An injection needle (not illustrated) may be attached to the third port 33 to administer the PRP into an affected area of the patient.

### Example 8

PRP Preparation Method 3 using Preparation Kit 110f
A process of preparing PRP using the preparation kit 110f will be described with reference to FIG. 24. FIG. 24 is a diagram illustrating an example of a process flow for preparing PRP from blood by using the preparation kit 110f illustrated in FIG. 14.

State <1g> to State <6g>

Each of the states <1g> to <6g> indicates a state the same as or similar to each of the above-described states <1d> to <5d> and <6f except that the third buoy 53 is located inside the third syringe barrel 31.

State <7g> Extraction Processing

The third plunger 32 coupled to the third gasket 34 is pulled in a direction in which the third gasket 34 is pulled out of the third syringe barrel 31. As a result, the third gasket 34 moves, and the liquid of the heavy layer H2 is stored in the third syringe barrel 31. The movement of the third gasket 34 in the direction in which the third syringe barrel 31 is pulled out may be ended when the second buoy 52 comes into contact with the second gasket 24. Thus, the liquid of the heavy layer H2 in the second syringe barrel 21 may be selectively moved into the third syringe barrel 31. When the second buoy 52 comes into contact with the second gasket 24, the second buoy 52 may close the liquid channel (for example, the second attachment hole 241) provided in the second gasket 24 into the third syringe barrel 31. In this case, the liquid of the heavy layer H2 in the second syringe barrel 21 may be more selectively moved into the third syringe barrel 31. That is, the possibility of movement of the liquid of the light layer L2 in the second syringe barrel 21 into the third syringe barrel 31 may be lowered.

State <8g> Third Centrifugation Processing

The third syringe 30 is removed from the second gasket 24. After the third cap 36 is attached to the third port 33, the third syringe barrel 31 is used as a centrifugation container to perform the third centrifugation processing using centrifugal force directed from the third port 33 side toward the third gasket 34 side. By the third centrifugation processing, the liquid in the third syringe barrel 31 is fractionated into the heavy layer H3 containing white blood cells and the light layer L3 (PRP layer) containing platelets. When the third buoy 53 having a specific gravity smaller than the specific gravity of the heavy layer H3 and larger than the specific gravity of the light layer L3 is used, the third buoy 53 is located at a boundary portion between the heavy layer H3 and the light layer L3.

The liquid containing white blood cells (heavy layer H2) in the third syringe barrel 31 can be discharged from the third syringe barrel 31 by using the third plunger 32, thereby making it possible to prepare the amount of white blood cells. An injection needle (not illustrated) may be attached to the third port 33 to administer the PRP (light layer L3) into an affected area of the patient.

In the present disclosure, the invention has been described above based on the various drawings and examples. However, the invention according to the present disclosure is not limited to each embodiment described above. That is, the embodiments of the invention according to the present disclosure can be modified in various ways within the scope illustrated in the present disclosure, and embodiments obtained by appropriately combining the technical means disclosed in different embodiments are also included in the technical scope of the invention according to the present disclosure. In other words, a person skilled in the art can easily make various variations or modifications based on the present disclosure. Note that these variations or modifications are included within the scope of the present disclosure.

### REFERENCE SIGNS

10 First syringe
11 First syringe barrel
13 First port (first leading end portion)
14 First gasket
20 Second syringe
21 Second syringe barrel
23 Second port (second leading end portion)
24 Second gasket
30 Third syringe
31 Third syringe barrel
33 Third port (third leading end portion)
34 Third gasket
51 First buoy
52 Second buoy
53 Third buoy
90 First needle
91 First end
92 Second end
93 Second needle
94 Fourth end
95 Third end
95 First needle guide
96 Second needle guide
99 Injection needle
100, 100a to 100f Syringe system
110, 110a to 110f Adjustment Kit
511 First buoy member
512 Second buoy member
521 Third buoy member
522 Fourth buoy member

1. A syringe system comprising:
a first syringe comprising a first syringe barrel;
a second syringe comprising a second syringe barrel coupled to the first syringe in a movable manner from inside the first syringe barrel; and
at least one of a first buoy located inside the first syringe barrel or a second buoy located inside the second syringe barrel.

2. The syringe system according to claim 1, wherein
the first syringe further comprises a first gasket configured to be positioned inside the first syringe barrel, and
the second syringe comprises a second leading end portion configured to be coupled to the first gasket and moved from inside the first syringe barrel to inside the second syringe barrel.

3. The syringe system according to claim 1, further comprising a first needle, wherein
the first needle is a tubular needle comprising a first end configured to be inserted into a first gasket, the first gasket configured to be positioned inside the first syringe barrel, and a second end located on an opposite side to the first end, and
the second syringe barrel comprises a leading end portion configured to be coupled to the second end of the first needle.

4. The syringe system according to claim 1, comprising:
the second buoy located inside the second syringe barrel.

5. The syringe system according to claim 1, comprising:
the second buoy, wherein
the second buoy comprises two buoys having different masses per unit volume.

6. The syringe system according to claim 1, comprising:
the first buoy located inside the first syringe barrel.

7. The syringe system according to claim 6, wherein
the first buoy comprises two buoys having different masses per unit volume.

8. The syringe system according to claim 1, comprising:
the first buoy and the second buoy.

9. The syringe system according to claim 8, wherein
a mass per unit volume of the first buoy is heavier than a mass per unit volume of the second buoy.

10. The syringe system according to claim 1, further comprising:
a third syringe comprising a third syringe barrel, wherein
the second syringe and the third syringe are configured to be coupled from inside the second syringe barrel to inside the third syringe barrel.

11. The syringe system according to claim 10, wherein
the third syringe comprises at least one third buoy located inside the third syringe barrel.

12. The syringe system according to claim 11, wherein
a mass per unit volume of the first buoy is heavier than a mass per unit volume of the third buoy.

13. The syringe system according to claim 11, wherein
a mass per unit volume of the second buoy is lighter than a mass per unit volume of the third buoy.

14. The syringe system according to claim 10, wherein
at least part of the third syringe is configured to be positioned inside the second syringe barrel.

15. The syringe system according to claim 10,
wherein
an internal volume of the third syringe barrel is smaller than an internal volume of the second syringe barrel.

16. The syringe system according to claim 1, wherein
at least part of the second syringe is configured to be positioned inside the first syringe barrel.

17. The syringe system according to claim 1, wherein
the internal volume of the second syringe barrel is smaller than an internal volume of the first syringe barrel.

18. The syringe system according to claim 1, wherein
the first buoy has a substantially circular flat plate shape, a disk shape with a conical surface in which a center portion protrudes relative to a peripheral edge portion, or a disk shape with a curved surface in which a center portion protrudes relative to a peripheral edge portion.

19. The syringe system according to claim 1, wherein
the second buoy has a substantially circular flat plate shape, a disk shape with a conical surface in which the center portion protrudes relative to the peripheral edge portion, or a disk shape with a curved surface in which the center portion protrudes relative to the peripheral edge portion.

20. The syringe system according to claim 1, wherein
the first buoy comprises a first covering portion configured to cover at least part of the first gasket configured to be positioned inside the first syringe barrel.

21. The syringe system according to claim 1, wherein
the second buoy comprises a second covering portion configured to cover at least part of a second gasket configured to be positioned inside the second syringe barrel.

22. The syringe system according to claim 18, wherein
the first buoy comprises any of a through hole, a net-like portion, and a nonwoven fabric-like portion in at least one location.

23. The syringe system according to claim 19, wherein
the second buoy comprises any of a through hole, a net-like portion, and a nonwoven fabric-like portion in at least one location.

24. The syringe system according to claim 1, wherein
the first buoy and the second buoy comprise two or more buoy members having different masses per unit volume.

25. The syringe system according to claim 1, wherein
a mass per unit volume of the first buoy is heavier than a mass per unit volume of whole blood of a human.
